SEQUENCE LISTING (14 sequences)

SEQ ID NO: 1 - protein, 10 aa, synthetic construct
  REGION 1..10: note = CDRH1
GYSFTSYWIG

SEQ ID NO: 2 - protein, 10 aa, synthetic construct
  REGION 1..10: note = CDRH2
VIYPGDSYTR

SEQ ID NO: 3 - protein, 9 aa, synthetic construct
  REGION 1..9: note = CDR H3 SEQUENCE
MPNWGSLDH

SEQ ID NO: 4 - protein, 12 aa, synthetic construct
  REGION 1..12: note = CDRL1
RASQSISSSYLA

SEQ ID NO: 5 - protein, 7 aa, synthetic construct
  REGION 1..7: note = CDRL2
GASSRAT

SEQ ID NO: 6 - protein, 5 aa, synthetic construct
  REGION 1..5: note = CDRL3 SEQUENCE
QQYAS

SEQ ID NO: 7 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSS

SEQ ID NO: 8 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEI

SEQ ID NO: 9 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 11 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 13 - protein, 443 aa, synthetic construct
  REGION 1..443: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 14 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0325475 A1
(43) Pub. Date: Oct. 23, 2025

(54) ANTI-IL-13R ANTIBODY FORMULATION

(71) Applicant: Aslan Pharmaceuticals Pte Ltd, Singapore (SG)

(72) Inventors: Alan LUK, Millbrae, CA (US); Robert MOORE, Singapore (SG); Danny CHOU, Lomita, CA (US); Robin HWANG, Thousand Oaks, CA (US); Nils KRAUSE, Munich (DE); Angelika REICHEL, Zwickau (DE)

(21) Appl. No.: 18/705,569

(22) PCT Filed: Oct. 28, 2022
(86) PCT No.: PCT/SG2022/050779

§ 371 Date: Apr. 28, 2024

Related U.S. Application Data
(60) Provisional application No. 63/332,737, filed on Apr. 20, 2022.

(30) Foreign Application Priority Data
Oct. 29, 2021 (SG) 10202112096R
Aug. 29, 2022 (SG) 10202250847P

(51) Int. Cl. A61K 9/08; A61K 47/02; A61K 47/18; A61K 47/22; A61K 47/26; C07K 16/24

(52) U.S. Cl. CPC A61K 9/08; A61K 47/02; A61K 47/183; A61K 47/22; A61K 47/26; C07K 16/244

(57) ABSTRACT

A stable formulation of an anti-IL 13R antibody. Also provided is the use of this formulation for treatment, method of treatment using the formulation and a process of making the formulation.

### CROSS-REFERENCE TO RELATED APPLICATIONS

This application is a national stage application, filed under 35 U.S.C. § 371, of International Patent Application No. PCT/SG2022/050779 filed on Oct. 28, 2022, which claims priority to SG 10202112096R filed on Oct. 29, 2021, U.S. 63/332,737 filed on Apr. 20, 2022, and SG 10202250847P filed on Aug. 29, 2022, the content of each of which applications is incorporated herein by reference.

### REFERENCE TO AN ELECTRONIC SEQUENCE LISTING

The instant application contains a Sequence Listing submitted via EFS-Web, hereby incorporated by reference in its entirety, which was created on Apr. 4, 2024, is named “ST.APL30.NP_seqlist.xml” and is 20,940 bytes in size and corresponds to the Sequence Listing originally submitted in International Appl. No. PCT/SG2022/050779.

The present disclosure relates to a formulation of an anti-IL13R antibody and uses thereof, a method of treatment using the formulation, in particular for treating a condition disclosed herein, and a process of making the formulation.

### BACKGROUND

IL-13 has been associated with various conditions, including but not limited to various respiratory and allergy-mediated disorders, fibrosis, scleroderma, atopic dermatitis, inflammatory bowel disease and certain cancers; see, e.g., Wynn, T. A., 2003 Annu. Rev. Immunol. 21:425-456; Terabe et al, 2000 Nat. Immunol. 1 (6): 515-520; Fuss et al, 2004 J. Clin. Invest. 113 (10): 1490-1497; Simms et al, 2002 Curr. Opin. Rheumatol. 14 (6): 717-722; and Hasegawa et al, 1997 J. Rheumatol. 24 (2): 328-332. Thus, IL-13 is an attractive target for the treatment of such diseases.

One possible way to inhibit the activity of IL-13 would be to interfere with the binding of IL-13 to its receptor IL-13R, for example by using an antibody specific to IL-13R, such as an antibody specific to IL-13Rαl. An effective antibody antagonist to IL-13Rαl may also interfere with the binding of IL-13 and prevent heterodimerization of IL-4Rα and IL-13Rαl. Such an antibody could inhibit signaling of both IL-13 and IL-4 through the type II receptor while sparing IL-4 signalling through the type I receptor. Signalling through the type I receptor is essential in the induction phase of the immune response during which Th2 cells differentiate. T cells do not express IL-13Rαl so the type II receptor plays no role in Th2 differentiation. Hence, an IL-13Rαl antibody should not affect the overall Th1/Th2 balance. Signalling through the type II IL-4/IL-13 receptor is critical during the effector-A-stage of the immune response during established allergic inflammation. Thus, blockade of the type II receptor should have a beneficial effect on many of the symptoms of asthma and other IL-13R-mediated conditions and should, therefore, be an effective disease modifying agent.

Antibodies against IL-13Rαl (both monoclonal and polyclonal) have been described in the art; see, eg, WO 97/15663, WO 03/80675; WO 03/46009; WO 06/072564; Gauchat et al, 1998 Eur. J. Immunol. 28:4286-4298; Gauchat et al, 2000 Eur. J. Immunol. 30:3157-3164; Clement et al, 1997 Cytokine 9 (11): 959 (Meeting Abstract); Ogata et al, 1998 J. Biol. Chem. 273:9864-9871; Graber et al, 1998 Eur. J. Immunol. 28:4286-4298; C. Vermot-Desroches et al, 2000 Tissue Antigens 5 (Supp. 1): 52-53 (Meeting Abstract); Poudrier et al, 2000 Eur. J. Immunol. 30:3157-3164; Akaiwa et al, 2001 Cytokine 13:75-84; Cancino-Diaz et al, 2002 J. Invest. Dermatol. 119:1114-1120; and Krause et al, 2006 Mol. Immunol. 43:1799-1807.

One particularly promising anti-IL-13Rαl antibody is CSL334 (now known as ASLAN004/eblasakimab), described in WO2008/060813 as antibody 10G5-6. Eblasakimab has been shown to bind to human IL-13Rαl with a high affinity (for example Kd may be 500 pM). Eblasakimab was shown to effectively antagonise IL-13 function through inhibiting the binding of IL-13 to its receptor IL-13Rαl and to inhibit IL-13 and IL-4 induced eotaxin release in NHDF cells, IL-13 and IL-4 induced STAT6 phosphorylation in NHDF cells and IL-13 stimulated release of TARC in blood or peripheral blood mononuclear cells.

However, an optimised formulation for eblasakimab is required because it can be difficult to handle and manufacture. Creating high concentration antibody formulations, for example with an antibody/fragment concentration of greater than 150 mg/ml is beneficial for patients because it allows smaller injection volumes but is not easy to achieve. Formation of aggregates and particles in parenteral formulations can be extremely dangerous to patients and must be avoided. Thus, optimised formulations are needed to address these problems and/or also to maximise the shelf life, delivery, potency and efficacy of the same.

### SUMMARY OF THE DISCLOSURE

The present disclosure is summarised in the following paragraphs:
1. A high concentration antibody formulation for thereof, said formulation comprising:
   150 to 210 mg/ml of an anti-IL-13R antibody or antigen binding fragment thereof, for example 150, 155, 160, 165, 170, 175, 180, 185, 190, 195 or 200 mg/ml, in particular 150 mg/ml, 175 mg/ml or 200 mg/ml;
   170 to 250 mM of arginine (such as Arg-HCl or Arg-Glu), for example 170, 175, 180, 185, 190, 195, 200, 205, 210, 215, 220, 225, 230, 235, 240, 245 or 250 mM, in particular 150 mM, 175 mM or 250 mM;
   20 to 50 mM histidine buffer, for example 20, 25, 30, 35, 40, 45 or 50 mM, such as 20 mM or 50 mM histidine buffer;
   0.01-0.03% of a non-ionic surfactant, for example 0.01-0.03% w/w, such as 0.02% w/w; wherein the pH of the formulation is in the range 6.0 to 7.0, such as 6.0, 6.1, 6.2, 6.3, 6.4, 6.5, 6.6, 6.7, 6.8, 6.9 or 7.0, in particular 6.5; and
   wherein the anti-IL-13R antibody or antigen binding fragment thereof comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: 3; and comprises a VL CDR1 comprising an amino acid sequence as set forth in SEQ ID NO: 4, a VL CDR2 comprising an amino acid sequence as set forth in SEQ ID NO: 5, and a VL CDR3 comprising an amino acid sequence as set forth in SEQ ID NO: 6.
2. The formulation according to paragraph 1, wherein the formulation comprises 150 to 200 mg/ml, such as 150, 155, 160, 165, 170, 175, 180, 185, 190, 195 or 200 mg/ml of the anti-IL13R antibody or a binding fragment thereof.
3. The formulation according to paragraphs 1 or 2, wherein the formulation comprises 150, 175 or 200 mg/ml of antibody.
4. The formulation according to paragraph 3, comprising 150 mg/ml.
5. The formulation according to paragraph 3, comprising 175 mg/ml.
6. The formulation according to paragraph 3, comprising 200 mg/ml.
7. The formulation according to any one of paragraphs 1 to 6, wherein the formulation comprises 175, 200 or 250 mM arginine.
8. The formulation according to any one of paragraphs 1 to 7, wherein the arginine is Arg-HCl.
9. The formulation according to any one of paragraphs 1 to 8, wherein the formulation comprises 20 mM histidine buffer.
10. The formulation according to any one of paragraphs 1 to 9, wherein the formulation further comprises phenylalanine, such as 45 to 85 mM phenylalanine.
11. The formulation according to paragraph 10, wherein the formulation comprises 50, 75 or 80 mM phenylalanine.
12. The formulation according to any one of paragraphs 1 to 11, wherein the formulation further comprises CaCl₂), for example 30, 35, 40, 45, 50, 55, 60, 65 or 70 mM CaCl₂).
13. The formulation according to paragraph 12, wherein the formulation comprises 50 mM CaCl₂).
14. The formulation according to any one of paragraphs 1 to 13, wherein the osmolarity of the formulation is in the range 350 to 550 mOsmo/kg, for example 350, 355, 365, 370, 375, 380, 385, 390, 395, 400, 405, 410, 415, 420, 425, 430, 435, 440, 445, 450, 455, 460,465, 470, 475, 480, 485, 490, 495, 500, 505, 515, 520, 525, 530, 535, 540, 545, 550, such as 405 to 435 mOsmo/kg.
15. The formulation according to any one of paragraphs 1 to 14, which further comprises 50 to 200 mM of a sugar, for example 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, such as 180 mM sugar.
16. The formulation according to paragraph 15, wherein the formulation comprises 180 mM sugar.
17. The formulation according to paragraphs 15 or 16, wherein the sugar is selected from mannitol, sorbitol, dextrose, galactose, fructose, lactose, trehalose and sucrose.
18. The formulation according to paragraph 17, wherein the sugar is sucrose.
19. The formulation according to any one of paragraphs 1 to 18, comprising 0.02% w/w of a non-ionic surfactant.
20. The formulation according to any one of paragraphs 1 to 19, wherein the non-ionic surfactant is polysorbate, for example polysorbate 20, 40, 60, or 80, polysorbate 20.
21. The formulation according to paragraph 20, wherein the non-ionic surfactant is polysorbate 20.
22. The formulation according to any one of paragraphs 1 to 21, wherein the formulation does not comprise NaCl.
23. The formulation according to any one of paragraphs 1 to 21, wherein the formulation comprises 50 to 150 mM of NaCl, for example 50, 55, 60, 65, 70, 75, 80, 85, 90, 95, 100, 105, 110, 115, 120, 125, 130, 135, 140, 145, 150, such as 62.5 or 140 mM NaCl.
24. The formulation according to any one of paragraphs 1 to 23, wherein the formulation has a viscosity in the range of 10 to 30 cP (mPa·s), for example 10, 11, 12, 13, 14, 15, 16, 17, 18, 19, 20, 21, 22, 23, 24, 25, 26, 27, 28, 29 or 30 cP, such as 15 to 25 cP, in particular 20 cP.
25. The formulation according to any one of paragraphs 1 to 24, wherein the anti-IL-13R antibody is an anti-IL13Rα1 antibody.
26. The formulation according to any one of paragraphs 1 to 25, wherein the anti-IL-13R antibody binds to the epitope FFYQ.
27. The formulation according to any one of paragraphs 1 to 26, wherein the anti-IL-13R antibody comprises a VH domain comprising an amino acid sequence shown in SEQ ID NO: 7 or a sequence at least 95% identical thereto.
28. The formulation according to any one of paragraphs 1 to 27, wherein the anti-IL-13R antibody comprises a VL domain comprising an amino acid sequence shown in SEQ ID NO: 8 or a sequence at least 95% identical thereto.
29. The formulation according to any one of paragraphs 1 to 28, wherein the anti-IL13R antibody comprises a VH domain comprising an amino acid sequence shown in SEQ ID NO: 7 or a sequence at least 95% identical thereto and a VL domain comprising an amino acid sequence shown in SEQ ID NO: 8 or a sequence at least 95% identical thereto.
30. The formulation according to any one of paragraphs 1 to 29, wherein the anti-IL13R antibody comprises a VH domain comprising an amino acid sequence shown in SEQ ID NO: 7 and a VL domain comprising an amino acid sequence shown in SEQ ID NO: 8.
31. The formulation according to any one of paragraphs 1 to 30, wherein the anti-IL13R antibody is a human antibody.
32. The formulation according to any one of paragraphs 1 to 31, wherein the formulation comprises: 175 mg/ml of an anti-IL-13R antibody or antigen binding fragment thereof; 250 mM of arginine; 20 mM histidine buffer; 0.02% of a non-ionic surfactant; and wherein the pH of the formulation is 6.5.
33. The formulation according to any one of paragraphs 1 to 32, comprising:
   175 mg/ml of an anti-IL-13R antibody or antigen binding fragment thereof; 250 mM of Arg-HCl; 20 mM histidine buffer; 0.02% polysorbate 20; and wherein the pH of the formulation is 6.5.
34. The formulation according to any one of paragraphs 1 to 33, comprising:
   175 mg/ml of an anti-IL-13R antibody or antigen binding fragment thereof; 250 mM of Arg-HCl; 20 mM histidine buffer; 0.02% polysorbate 20; phenylalanine, such as 45 to 85 mM phenylalanine; and wherein the pH of the formulation is 6.5.
35. The formulation according to any one of paragraphs 1 to 34, comprising: 175 mg/ml of an anti-IL-13R antibody or antigen binding fragment thereof; 250 mM of Arg-HCl; 20 mM histidine buffer; 0.02% polysorbate 20; 75 mM phenylalanine; and wherein the pH of the formulation is 6.5.
36. The formulation according to any one of paragraphs 1 to 35, comprising:
175 mg/ml of an anti-IL-13R antibody or antigen binding fragment thereof, wherein the anti-IL13R antibody comprises a VL domain comprising an amino acid sequence shown in SEQ ID NO: 8 or a sequence at least 95% identical thereto, and a VH comprising an amino acid sequence shown in SEQ ID NO: 7 or a sequence at least 95% identical thereto; 250 mM of Arg-HCl; 20 mM histidine buffer; 0.02% polysorbate 20; 75 mM phenylalanine and wherein the pH of the formulation is 6.5.
37. The formulation according to any one of paragraphs 1 to 36, for use in treatment.
38. The formulation according to paragraph 37, for use in the treatment of inflammation or an autoimmune disease, for example chronic inflammation.
39. The formulation according to paragraph 38, wherein the inflammation is selected from the group comprising: fibrosis (including pulmonary fibrosis, such as cystic fibrosis, idiopathic pulmonary fibrosis, progressive massive fibrosis; liver fibrosis, such as cirrhosis; heart disease, such as atrial fibrosis, endomyocardial fibrosis, old myocardial infarction; arthrofibrosis; Dupuytren's contracture; keloid fibrosis; mediastinal fibrosis; myelofibrosis; nephrogenic systemic fibrosis; retroperitoneal fibrosis; and scleroderma) Hodgkin's disease, ulcerative colitis, Chron's disease, atopic dermatitis, eosinophilic esophagitis, allergic rhinitis, asthma and chronic pulmonary disease (including chronic obstructive pulmonary disease), in particular asthma.
40. Use of a formulation according to any one of paragraphs 1 to 36, for use in the manufacture of a medicament for the treatment of inflammation or an autoimmune disease, for example chronic inflammation.
41. Use of a formulation according paragraph 40 in the manufacture of a medicament for the treatment of a condition selected from the group comprising fibrosis (including pulmonary fibrosis, such as cystic fibrosis, iodiopathic pulmonary fibrosis, progressive massive fibrosis; liver fibrosis, such as cirrhosis; heart disease, such as atrial fibrosis, endomyocardial fibrosis, old myocardial infarction; arthrofibrosis; Dupuytren's contracture; keloid fibrosis; mediastinal fibrosis; myelofibrosis; nephrogenic systemic fibrosis; retroperitoneal fibrosis; and scleroderma) Hodgkin's disease, ulcerative colitis, Chron's disease, atopic dermatitis, eosinophilic esophagitis, allergic rhinitis, asthma and chronic pulmonary disease (including chronic obstructive pulmonary disease), Sezary syndrome, in particular asthma.
42. A method of treatment comprising administering an effective amount of a formulation according to any one of paragraphs 1 to 36.
43. A method of treating inflammation (such as chronic inflammation) or an autoimmune disease comprising administering an effective amount of a formulation according to any one of paragraphs 1 to 36.
44. The method according to paragraph 43, wherein the treatment of a condition selected from the group comprising: group comprising: fibrosis (including pulmonary fibrosis, such as cystic fibrosis, idiopathic pulmonary fibrosis, progressive massive fibrosis; liver fibrosis, such as cirrhosis; heart disease, such as atrial fibrosis, endomyocardial fibrosis, old myocardial infarction; arthrofibrosis; Dupuytren's contracture; keloid fibrosis; mediastinal fibrosis; myelofibrosis; nephrogenic systemic fibrosis; retroperitoneal fibrosis; and scleroderma) Hodgkin's disease, ulcerative colitis, Chron's disease, atopic dermatitis, eosinophilic esophagitis, allergic rhinitis, asthma and chronic pulmonary disease (including chronic obstructive pulmonary disease), in particular asthma.

Antibodies, such as eblasakimab need to be formulated to high concentration to allow the desired dose in man to be administered in the smallest possible volume. High concentration formulations pose unique challenges as phenomena like phase separation can be observed. Aggregation is also a common feature at high antibody concentration. However, the formulation needs to contain very high levels of antibody molecules as “monomer”, for example 99% monomer or more. In addition, the formulation needs to be stable when stored. Eblasakimab seems to have a hydrophobic portion in the protein, which for example interacts with hydrophobic interaction columns in the absence of high salt concentrations. This hypothesised hydrophobic portion adds additional complexity when formulating the antibody and preventing aggregation. Thus, the antibodies of the present disclosure are particularly difficult to formulate.

In addition, a difficult challenge presented by higher concentration antibody formulations is the tendency for such formulations to be overly viscous. Hence, there is a need to optimise formulations in order to keep viscosity at an acceptably low level, i.e. close to a target viscosity range of 20-25 cP, such as 20 cP.

The present inventors have optimised the formulation of the present disclosure and established that the IL-13R antibodies, such as eblasakimab, are most suitable for formulation within a narrow set of parameters. The formulations of the present disclosure are highly monomeric, for example at least 98% monomeric (such as 98 to 99.5% monomeric) even when formulated with high antibody concentration. The formulations of the present disclosure also have good viscosity at high antibody concentrations. In addition, the formulation is suitably stable, for example in some embodiments no change in monomer or less than a 0.5% reduction in monomer was observed when stored at 4° C. or 25° for 90 days. Accelerated ‘stress test’ studies at 40° C. also show the formulations of the present disclosure to be stable over a period of 60 days, for example using potency measurements.

The combination of features of the formulation of the present disclosure, including the pH, contributing to stabilising the IL-13 receptor antibody or binding fragment thereof.

In one embodiment the formulations of the present disclosure have a viscosity in the range of 10 to 30, such as 11, 12, 13, 14, 15, 16, 17, 18, 19, 20, 21, 22, 23, 24, 25, 26, 27, 28, 29 or 30 cP (centipoise), such as 20 cP, for example at ambient temperature. Surprisingly, the viscosity of the formations of the present disclosure are relatively low even at high concentrations of antibody.

In one embodiment, the viscosity is measured using a viscometer, such as rotational viscometer, an electromagnetically spinning-sphere (EMS) viscometer, or a Stabinger viscometer. In one embodiment the viscosity is measured using a rheometer, such as shear rheometer, dynamic shear rheometer, an extensional rheometer, a capillary rheometer. In one embodiment the viscosity is measured using a Kinexus-ultra+ rheometer (Netzsch).

In one embodiment the osmolarity of the formulation is in the range 350 to 450 mOsmo/kg, such as 390 to 430 mOsmo/kg, in particular 410+/−5 mOsmo/kg.

In one embodiment the formulation comprises 150 to 210 mg/ml or an anti-IL13R antibody, for example 150 to 175 mg/ml, such as 150, 155, 160, 165, 170, 175 mg/ml. In embodiment, the formulation comprises 175 mg/ml to 210 mg/ml, such as 175, 180, 185, 190, 195, 200, 205 or 210 mg/ml. In one embodiment the formulation comprises 150 mg/ml of anti-IL13R antibody. In another embodiment, the formulation comprises 175 mg/ml of anti-IL13R antibody. In one embodiment, the formulation comprises 200 mg/ml.

In one embodiment, the formulation comprises 170 to 260 mM of arginine, for example 170, 175, 180, 185, 190, 195, 200, 205, 210, 215, 220, 225, 230, 235, 240, 245, 250 or 260 mM. In one embodiment, the formulation comprises 150 mM, 175 mM, 200 mM or 250 mM arginine. In one embodiment the formulation comprises 150 mM arginine. In one embodiment the formulation comprises 175 mM arginine. In one embodiment the formulation comprises 200 mM arginine. In one embodiment the formulation comprises 250 mM arginine.

In one embodiment the arginine is Arg-HCl. In another embodiment, the arginine is Arg-Glu. In one embodiment arginine is L-arginine. Hence, in one embodiment the formulation comprises 150 mM Arg-HCl. In one embodiment the formulation comprises 175 mM Arg-HCl. In one embodiment the formulation comprises 200 mM Arg-HCl. In one embodiment the formulation comprises 250 mM Arg-HCl.

In one embodiment the formulation comprises 20 to 50 mM histidine buffer, for example 20, 25, 30, 35, 40, 45 or 50 mM, such as 20 mM or 50 mM histidine buffer. In one embodiment the formulation comprises 20 mM histidine buffer. In another embodiment the formulation comprises 50 mM histidine buffer.

In one embodiment the formulation comprises 0.01-0.03% of a non-ionic surfactant, such as 0.01, 0.015, 0.02, 0.025 or 0.030%, in particular 0.02%. In one embodiment the formulation comprises 0.01-0.03%, such as 0.01, 0.015, 0.02, 0.025 or 0.030%, in particular 0.02% volume per volume (v/v) of a non-ionic surfactant. In one embodiment the formulation comprises 0.01-0.03%, such as 0.01, 0.015, 0.02, 0.025 or 0.030%, in particular 0.02% weight per volume (w/v) of a non-ionic surfactant. In one embodiment the formulation comprises 0.01-0.03%, such as 0.01, 0.015, 0.02, 0.025 or 0.030%, in particular 0.02% weight per weight (w/w) of a non-ionic surfactant. In one embodiment the formulation comprises 0.02% w/w of a non-ionic surfactant.

In one embodiment the non-ionic surfactant is polysorbate, such as polysorbate 20, 40, 60, or 80.

In one embodiment the non-ionic surfactant is polysorbate 20. Thus, in one embodiment the formulation comprises 0.01-0.03%, such as 0.02% polysorbate 20 (for example as % w/w, % w/v, % v/w or % v/v). In one embodiment the formulation comprises 0.02% w/w polysorbate 20.

In one embodiment wherein the pH of the formulation is in the range 6.0 to 7.0, such as 6.0, 6.1, 6.2, 6.3, 6.4, 6.5, 6.6, 6.7, 6.8, 6.9 or 7.0. In one embodiment the pH is 6.0, 6.5 or 7.0. In one embodiment, the pH is 6.5.

In one embodiment the formulation further comprises phenylalanine, such as 45 to 90 mM phenylalanine, for example 45, 50, 55, 60, 65, 70, 75, 80, 85 or 90 mM. In one embodiment the formulation comprises 50, 75 or 80 mM phenylalanine. Thus, in one embodiment the formulation comprises 50 mM phenylalanine. In one embodiment the formulation comprises 75 mM phenylalanine. In one embodiment the formulation comprises 80 mM phenylalanine.

In one embodiment the formulation further comprises CaCl₂), for example 10, 20, 30, 40, 50 or 60 mM CaCl₂). In one embodiment the formulation comprises 50 mM CaCl₂).

In one embodiment the formulation further comprises 50 to 200 mM of a sugar, such as 50, 60, 70, 80, 90, 100, 110, 120, 130, 140, 150, 160, 170, 180, 190 or 200 mM of a sugar. In one embodiment the formulation comprises 180 mM of a sugar. In one embodiment the sugar is selected from mannitol, sorbitol, dextrose, galactose, fructose, lactose, trehalose and sucrose. In one embodiment the sugar is sucrose. Thus in one embodiment the formulation comprises 180 mM sucrose.

In one embodiment certain formulations of the present disclosure have 1% or less protein aggregation, for example when stored for 90 days at temperature in the range 2 to 25° C. The presently disclosed anti-IL13R antibody formulation is particularly suitable for stable long-term storage of the anti-IL13R antibody. In one embodiment the formulation is stored at a temperature in the range 2 to 8° C., such as 2, 3, 4, 5, 6, 7 or 8° C., such as 4° C.

In one embodiment there is provided a parenteral formulation (in particular a liquid formulation) for example for infusion or injection. In one embodiment there is provided liquid parenteral formulation as a concentrate for dilution with a liquid for injection, such as glucose, saline or water for injection. In one embodiment the liquid parenteral formulation is provided in a final concentration for administration without dilution, for example for injection or for infusion.

In one embodiment the formulation comprises: 175 mg/ml of an anti-IL-13R antibody or antigen binding fragment thereof; 250 mM of arginine; 20 mM histidine buffer; 0.02% of a non-ionic surfactant; and wherein the pH of the formulation is 6.5.

In one embodiment the formulation comprises: 175 mg/ml of an anti-IL-13R antibody or antigen binding fragment thereof; 250 mM of Arg-HCl; 20 mM histidine buffer; 0.02% of polysorbate 20; and wherein the pH of the formulation is 6.5.

In one embodiment the formulation comprises: 175 mg/ml of an anti-IL-13R antibody or antigen binding fragment thereof; 250 mM of arginine; 20 mM histidine buffer; 0.02% of a non-ionic surfactant; phenylalanine, such as 45 to 85 mM phenylalanine; and wherein the pH of the formulation is 6.5.

In one embodiment the formulation comprises: 175 mg/ml of an anti-IL-13R antibody or antigen binding fragment thereof; 250 mM of arginine; 20 mM histidine buffer; 0.02% of a non-ionic surfactant; 75 mM phenylalanine; and wherein the pH of the formulation is 6.5.

In one embodiment the formulation comprises: 175 mg/ml of an anti-IL-13R antibody or antigen binding fragment thereof; 250 mM of Arg-HCl; 20 mM histidine buffer; 0.02% of polysorbate 20; 75 mM phenylalanine; and wherein the pH of the formulation is 6.5.

In one embodiment the antibody or binding fragment employed in the formulation of the present disclosure is monoclonal.

In one embodiment the antibody or binding fragment employed in the formulation of the present disclosure is human. In one embodiment the antibody or binding fragment employed in the formulation of the present disclosure is chimeric or humanised.

In one embodiment the antibody or binding fragment thereof comprises a variable heavy region comprising a CDRH1 with a sequence shown in SEQ ID NO: 1, a CDRH2 with a sequence shown in SEQ ID NO: 2, and a CDRH3 with a sequence shown in SEQ ID NO: 3; and a variable light region comprising CDRL1 with a sequence shown in SEQ ID NO: 4, a CDRL2 with a sequence shown in SEQ ID NO: 5, and a CDRL3 with a sequence shown in SEQ ID NO: 6.

In one embodiment the antibody or binding fragment thereof comprises a VH domain comprising a sequence shown in SEQ ID NO: 7 or a sequence at least 95% identical thereto. In one embodiment the antibody or binding fragment thereof comprises a VL domain comprising a sequence shown in SEQ ID NO: 8 or a sequence at least 95% identical thereto.

In one embodiment the antibody or binding fragment thereof comprises a VH domain comprising a sequence shown in SEQ ID NO: 7 or a sequence at least 95% identical thereto and a VL domain comprising a sequence shown in SEQ ID NO: 8 or a sequence at least 95% identical thereto.

In one embodiment the antibody or binding fragment thereof comprises a VH domain comprising a sequence shown in SEQ ID NO: 7 and a VL domain comprising a sequence shown in SEQ ID NO: 8.

In one embodiment the antibody or binding fragment thereof is eblasakimab.

In one embodiment the formulation comprises: 175 mg/ml of an anti-IL-13R antibody or antigen binding fragment thereof comprising a VH domain comprising a sequence shown in SEQ ID NO: 7 or a sequence at least 95% identical thereto and a VL domain comprising a sequence shown in SEQ ID NO: 8;

250 mM of arginine; 20 mM histidine buffer; 0.02% of a non-ionic surfactant; and wherein the pH of the formulation is 6.5.

In one embodiment the formulation comprises: 175 mg/ml of an anti-IL-13R antibody or antigen binding fragment thereof comprising a VH domain comprising a sequence shown in SEQ ID NO: 7 or a sequence at least 95% identical thereto and a VL domain comprising a sequence shown in SEQ ID NO: 8; 250 mM of Arg-HCl; 20 mM histidine buffer; 0.02% of polysorbate 20; and wherein the pH of the formulation is 6.5.

In one embodiment the formulation comprises: 175 mg/ml of an anti-IL-13R antibody or antigen binding fragment thereof comprising a VH domain comprising a sequence shown in SEQ ID NO: 7 or a sequence at least 95% identical thereto and a VL domain comprising a sequence shown in SEQ ID NO: 8; 250 mM of arginine; 20 mM histidine buffer; 0.02% of a non-ionic surfactant; phenylalanine, such as 45 to 85 mM phenylalanine; and wherein the pH of the formulation is 6.5.

In one embodiment the formulation comprises: 175 mg/ml of an anti-IL-13R antibody or antigen binding fragment thereof comprising a VH domain comprising a sequence shown in SEQ ID NO: 7 or a sequence at least 95% identical thereto and a VL domain comprising a sequence shown in SEQ ID NO: 8; 250 mM of arginine; 20 mM histidine buffer; 0.02% of a non-ionic surfactant; 75 mM phenylalanine; and wherein the pH of the formulation is 6.5.

In one embodiment the formulation comprises: 175 mg/ml of an anti-IL-13R antibody or antigen binding fragment thereof comprising a VH domain comprising a sequence shown in SEQ ID NO: 7 or a sequence at least 95% identical thereto and a VL domain comprising a sequence shown in SEQ ID NO: 8; 250 mM of Arg-HCl; 20 mM histidine buffer; 0.02% of polysorbate 20; 75 mM phenylalanine; and wherein the pH of the formulation is 6.5.

In one embodiment the formulation comprises: 175 mg/ml of an anti-IL-13R antibody or antigen binding fragment thereof comprising a VH domain with a sequence shown in SEQ ID NO: 7 or a sequence at least 95% identical thereto and a VL domain comprising a sequence shown in SEQ ID NO: 8; 250 mM of Arg-HCl; 20 mM histidine buffer; 0.02% of polysorbate 20; 75 mM phenylalanine; and wherein the pH of the formulation is 6.5.

### DETAILED DISCLOSURE

Long term as used herein refers to a period of at least 6 months, such as 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months. In one embodiment, the disclosed formulation storage for at least 12 months, such as 12 months, 18 months and 24 months.

Non-ionic surfactant as employed herein refers to surfactants that have covalently bonded oxygen-containing hydrophilic groups which are bonded to hydrophobic parent structures. Examples of non-ionic surfactants include ethoxylates, such as fatty alcohol ethoxylates (such as narrow-range ethoxylate, octaethylene glycol monododecyl ether and pentaethylene glycol monododecyl ether), alkylphenol ethoxylates (such as nonoxynols and Triton X-100), fatty acid ethoxylates, ethoxylated amines and/or fatty acid amides (such as polyethoxylated tallow amine, cocamide monoehtnolamine and cocamide diethanolamine), terminally blocked ethoxylates (such as poloxamers); fatty acid esters of polyhydroxycompounds; fatty acid esters of glycerol (such as glycerol monostearate and glycerol monolaurate); fatty acid esters of sorbitol (such as sorbitan monolaurate, sorbitant monosterate and sorbitan tristearate; Tweens such as Tween 20, 40, 60 or 80); fatty acid esters of sucrose; alkyl polyglucosides (such as decyl glucoside, lauryl glucoside and octyl glucose); and polysorbates (such as polysorbate 20, 40, 60 or 80).

Thus, in one embodiment the non-ionic surfactant is selected from the group comprising ethoxylates; fatty acid esters of polyhydroxy compounds; fatty acid esters of glycerol; fatty acid esters of sorbitol; Tweens; fatty acid esters of sucrose; alkyl polyglucosides; and polysorbates.

Parenteral formulation as employed herein refers to a formulation designed not to be delivered through the GI tract. Typical parenteral delivery routes include injection (including bolus injection), implantation or infusion. In one embodiment the formulation is provided in a form for bolus delivery.

In one embodiment the parenteral formulation is administered intravenously. In one embodiment the parenteral formulation is administered subcutaneously.

Injection as employed herein refers to the administration of a liquid formulation into the body via a syringe or syringe driver. Injection includes intravenous, subcutaneous, intra-tumoral or intramuscular administration. The injection is generally over a short period of time, such as 5 minutes or less. However, injection can be administered slowly or continuously, for example using a syringe driver. Injections generally involve administration of smaller volumes than infusions. In one embodiment the injection is administered as a slow injection, for example over a period of 1.5 to 30 minutes. Slow injection as employed herein is manual injection with syringe. In one embodiment one dose of the formulation less than 100 mls, for example 30 mls, such as administered by a syringe driver.

Infusion as employed herein means the administration of fluids by drip, infusion pump, or equivalent device. In one embodiment the infusion is administered over a period in the range of 1 to 120 minutes (for example 1 to 5 minutes), such as about 1, 3, 4, 5, 6, 7, 8, 9, 10, 11, 12, 13, 14, 15, 16, 17, 18, 19 20, 25, 30, 35, 40, 45, 50, 55, 60, 65, 70, 65, 80, 85, 90, 95, 100, 105, 110, 115 or 120 minutes.

### Anti-IL13R Antibody

Interleukin-13 receptor (IL-13R) as used herein is a type I cytokine receptor, which binds to Interleukin-13. It consists of two subunits, encoded by IL13Rα1 and IL4R, respectively. These two genes encode the proteins IL-13Rα1 and IL-4Rα. These form a dimer with IL-13 binding to the IL-13Rα1 chain and IL-4Rα stabilises this interaction. Due to the presence of the IL4R subunit, IL13R can also instigate IL-4 signalling. In both cases this occurs via activation of the Janus kinase (JAK)/Signal Transducer and Activator of Transcription (STAT) pathway, resulting in phosphorylation of STAT6. Human IL-13Rα1 has the Uniprot number P3597.

IL-13Rα2, previously called IL-13R and IL-13Rα, is another receptor which is able to bind to IL-13. However, in contrast to IL-13Rα1, this protein binds IL-13 with high affinity, but it does not bind IL-4. Human IL-13Rα2 has the Uniprot number Q14627.

Anti-IL13R antibody as used herein refers to an antibody that has specificity for IL13R, for example IL13Rα1 or IL13Rα2.

In one embodiment, the anti-IL13R antibody of the present disclosure is specific for IL13Rx1. In one embodiment, the anti-IL13R antibody binds to an epitope comprising the amino acid sequence FFYQ.

The anti-IL13R antibodies of the present disclosure may comprise a complete antibody molecule having full length heavy and light chains or a binding fragment thereof. Binding fragments include but are not limited to Fab, modified Fab, Fab′, F(ab′)₂, Fv, single domain antibodies (such as VH, VL, VHH, IgNAR V domains), scFv, bi, tri or tetra-valent antibodies, Bis-scFv, diabodies, triabodies, tetrabodies and epitope-binding fragments of any of the above (see for example Holliger and Hudson, 2005, Nature Biotech. 23 (9): 1126-1136; Adair and Lawson, 2005, Drug Design Reviews-Online 2 (3), 209-217).

The methods for creating and manufacturing these antibody fragments are well known in the art (see for example Verma et al, 1998, Journal of Immunological Methods, 216, 165-181). Other antibody fragments for use in the present invention include the Fab and Fab′ fragments described in WO2005/003169, WO2005/003170 and WO2005/003171. Other antibody fragments for use in the present invention include Fab-Fv and Fab-dsFv fragments described in WO2010/035012 and antibody fragments comprising those fragments. Multi-valent antibodies may comprise multiple specificities or may be monospecific (see for example WO 92/22853 and WO05/113605).

The antibody and fragments thereof, for use in the present disclosure may be from any species including for example mouse, rat, shark, rabbit, pig, hamster, camel, llama, goat or human. Chimeric antibodies have a non-human variable regions and human constant regions. An antibody or binding fragment for use in the present invention can be derived from any class (e.g. IgG, IgE, IgM, IgD or IgA) or subclass of immunoglobulin molecule. In one embodiment the antibody employed in the present disclosure is IgG4 or IgG4 with a 241P mutation.

In one embodiment the antibody or binding fragment employed in the formulation of the present disclosure has affinity of 5 nM or higher (higher affinity is a lower numerical value), for example 500 pM, such as 250 pM or higher, in particular 125 pM or less.

A sequence listing is filed herewith.

(SEQ ID NO: 1)
GYSFTSYWIG
(SEQ ID NO: 2)
VIYPGDSYTR
(SEQ ID NO: 3)
MPNWGSLDH
(SEQ ID NO: 4)
RASQSISSSYLA
(SEQ ID NO: 5)
GASSRAT
(SEQ ID NO: 6)
QQYAS
SEQ ID NO: 7
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
MPNWGSLDHWGQGTLVTVSS
SEQ ID NO: 8
EIVLTQSPGTLSLSPGERATLSCRASQSISSSYLAWYQQKPGQAPRLLI
YGASSRATGIPDRFSGSGSGTDFTLTISRLEPEDFAVYYCQQYASFGQG
TKVEI*
(*K deleted in a post translational modification)

EVQLVQSGAEVKKPGESLKISCKGSGYSFTSYWIGWVRQMPGKGLEWMGVIYPGDSYTRYSPSFQGQVTIS ADKSISTAYLQWSSLKASDTAMYYCARMPNWGSLDHWGQGTLVTVSS

SEQ ID NO: 8

EIVLTQSPGTLSLSPGERATLSCRASQSISSSYLAWYQQKPGQAPRLLIYGASSRATGIPDRFSGSGSGTDFTL TISRLEPEDFAVYYCQQYASFGQGTKVEI* (* K deleted in a post translational modification).

The anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 3.

The anti-IL13R antibody or binding fragment employed in the present disclosure comprises a CDRL1 comprising an amino acid sequence as set forth in SEQ ID NO: 4, a CDRL2 comprising an amino acid sequence as set forth in SEQ ID NO: 5, and a CDRL3 comprising an amino acid sequence as set forth in SEQ ID NO: 6.

In one embodiment the VH sequence comprises SEQ ID NO: 7 or a sequence at least 95% identical thereto. In one embodiment the VL sequence comprises SEQ ID NO: 8 or a sequence at least 95% identical thereto.

In one embodiment the VH sequence comprises SEQ ID NO: 7 or a sequence at least 95% identical thereto and the VL sequence comprises SEQ ID NO: 8 or a sequence at least 95% identical thereto.

In one embodiment the VH sequence is SEQ ID NO: 7 and the VL sequence is SEQ ID NO: 8.

Variable region as employed herein refers to the region in an antibody chain comprising the CDRs and a suitable framework.

In one embodiment the heavy chain comprises a sequence independently selected from the group comprising: SEQ ID NO: 9, SEQ ID NO: 10, SEQ ID NO: 11, SEQ ID NO: 12, SEQ ID NO: 13 and a sequence at least 95% identical to any one of the same.

In one embodiment the light chain comprises an amino acid sequence as set forth in SEQ ID NO: 14 or a sequence at least 95% identical thereto.

In one embodiment the heavy chain comprises SEQ ID NO: 9 or a sequence at least 95% identical thereto and the light chain comprises SEQ ID NO: 14 or a sequence at least 95% identical thereto.

In one embodiment the heavy chain comprises SEQ ID NO: 10 or a sequence at least 95% identical thereto and the light chain comprises SEQ ID NO: 14 or a sequence at least 95% identical thereto.

In one embodiment the heavy chain comprises SEQ ID NO: 11 or a sequence at least 95% identical thereto and the light chain comprises SEQ ID NO: 14 or a sequence at least 95% identical thereto.

In one embodiment the heavy chain comprises SEQ ID NO: 12 or a sequence at least 95% identical thereto and the light chain comprises SEQ ID NO: 14 or a sequence at least 95% identical thereto.

In one embodiment the heavy chain comprises SEQ ID NO: 13 or a sequence at least 95% identical thereto and the light chain comprises SEQ ID NO: 14 or a sequence at least 95% identical thereto.

In one embodiment the heavy chain is SEQ ID NO: 9 and the light chain is SEQ ID NO: 14. In one embodiment the heavy chain is SEQ ID NO: 10 and the light chain is SEQ ID NO: 14. In one embodiment the heavy chain is SEQ ID NO: 11 and the light chain is SEQ ID NO: 14. In one embodiment the heavy chain is SEQ ID NO: 12 and the light chain is SEQ ID NO: 14. In one embodiment the heavy chain is SEQ ID NO: 13 and the light chain is SEQ ID NO: 14.

Derived from as employed herein refers to the fact that the sequence employed or a sequence highly similar to the sequence employed was obtained from the original genetic material, such as the light or heavy chain of an antibody.

“At least 95% identical” as employed herein is intended to refer to an amino acid sequence which over its full length is 95% identical or more to a reference sequence, such as 96, 97, 98 or 99% identical. Software programmes can be employed to calculate percentage identity.

In one embodiment an antibody or binding fragment thereof, employed in a formulation of the present disclosure is humanised.

Humanised (which include CDR-grafted antibodies) as employed herein refers to molecules having one or more complementarity determining regions (CDRs) from a non-human species and a framework region from a human immunoglobulin molecule (see, for example U.S. Pat. No. 5,585,089; WO91/09967). It will be appreciated that it may only be necessary to transfer the specificity determining residues of the CDRs rather than the entire CDR (see for example, Kashmiri et al., 2005, Methods, 36, 25-34). Humanised antibodies may optionally further comprise one or more framework residues derived from the non-human species from which the CDRs were derived. For a review, see Vaughan et al, Nature Biotechnology, 16, 535-539, 1998.

When the CDRs or specificity determining residues are grafted, any appropriate acceptor variable region framework sequence may be used having regard to the class/type of the donor antibody from which the CDRs are derived, including mouse, primate and human framework regions. Examples of human frameworks which can be used in the present invention are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/In a humanised antibody employed in the present invention, the acceptor heavy and light chains do not necessarily need to be derived from the same antibody and may, if desired, comprise composite chains having framework regions derived from different chains.

The framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

In one embodiment the anti-IL13R antibodies of the present disclosure are fully human, in particular one or more of the variable domains are fully human.

Fully human molecules are those in which the variable regions and the constant regions (where present) of both the heavy and the light chains are all of human origin, or substantially identical to sequences of human origin, not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP0546073 B1, U.S. Pat. Nos. 5,545,806, 5,569,825, 5,625,126, 5,633,425, 5,661,016, 5,770,429, EP 0438474 and EP0463151.

Constant region as employed herein is intended to refer to the constant region portion located between two variable domains, for example non-cognate variable domains, in the heavy chain. Thus, the presently disclosed anti-IL13R antibody may comprise one or more constant regions, such as a naturally occurring constant domain or a derivate of a naturally occurring domain.

A derivative of a naturally occurring domain as employed herein is intended to refer to where one, two, three, four or five amino acids in a naturally occurring sequence have been replaced or deleted, for example to optimize the properties of the domain such as by eliminating undesirable properties but wherein the characterizing feature(s) of the domain is/are retained.

If desired an antibody for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO03031581. Alternatively, where the effector molecule is a protein or polypeptide the linkage may be achieved using recombinant DNA procedures, for example as described in WO 86/01533 and EP0392745.

The term effector molecule as used herein includes, for example, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 1251, 1311, 111 In and 99Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984. Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof. Specific naturally occurring polymers include lactose, amylose, dextran, glycogen or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

In patients with cancer, such as breast cancer, cancer related lymphedema (BCRL), the formulation of the present disclosure may prevent lymphedema-associated effects, such as fibrosis, hyperkeratosis, the deposition of fibroadipose tissue, fluid accumulation, limb swelling, reduction of skin elasticity, and pain. By reducing the excess volume, said formulation may improve lymphatic and, for example limb functions.

The development of lymphedema after lymphatic injury is associated with tissue inflammation, the infiltration of CD4-positive cells and their differentiation to the type 2 helper T-cell (Th2) phenotype. Th2 cells produce IL-4 and IL-13 that play a key role in the development of lymphedema-associated symptoms as well as other Th2-mediated diseases.

In one embodiment the formulation herein is administered in combination with another therapy. “In combination” as employed herein is intended to encompass where the anti-IL13R antibody is administered before, concurrently with, or after another therapy.

Therapeutic dose as employed herein refers to the amount of the anti-IL13R antibody, such as eblasakimab that is suitable for achieving the intended therapeutic effect when employed in a suitable treatment regimen, for example ameliorates symptoms or conditions of a disease, in particular without eliciting dose limiting side effects. Suitable therapeutic doses are generally a balance between therapeutic effect and tolerable toxicity, for example where the side-effect and toxicity are tolerable given the benefit achieved by the therapy.

In one embodiment a formulation according to the present disclosure (including a formulation comprising same) is administered monthly, for example in a treatment cycle or as maintenance therapy.

In the context of this specification “comprising” is to be interpreted as “including”. Embodiments of the invention comprising certain features/elements are also intended to extend to alternative embodiments “consisting” or “consisting essentially” of the relevant elements/features. Where technically appropriate, embodiments of the invention may be combined.

Corrections to the present specification may be based on the priority document(s).

The background section may be employed as basis to make amendments.

Technical references such as patents and applications are incorporated herein by reference.

Any embodiments specifically and explicitly recited herein may form the basis of a disclaimer either alone or in combination with one or more further embodiments.

Embodiments may be combined when technically appropriate.

Subject headings herein are employed to divide the document into sections and are not intended to be used to construe the meaning of the disclosure provided herein.

The present invention is further described by way of illustration only the following examples.

### BRIEF SUMMARY OF THE FIGURES

FIG. 1A shows a schematic diagram of the Kinexus ultra+ rheometer used for the viscosity measurements

FIG. 1B shows a graph of the viscosity of the eblasakimab bulk drug product (BDP)

FIG. 2 shows a series of graphs indicating the viscosity of Formulations 1 to 15

FIG. 3 shows a graph comparing the viscosity of formulations comprising different Arg-HCl concentrations

FIG. 4 shows a graph showing the viscosity of formulations comprising 150 mM Arg-HCl+ additional excipients

FIG. 5 shows graph comparing the viscosity of Formulations 1 to 15

FIG. 6 shows a comparison of viscosity measurements for eblasakimab BDP from the 1st and 2nd step screens

FIG. 7 shows a series of graphs comparing the viscosity of Formulation 3 and 6

FIG. 8 shows a graph of a repeat viscosity measurement for Formulation 3 to check reproducibility of viscosity measurements

FIG. 9 shows a series of graphs indicating the viscosity of Formulations 16 to 30

FIG. 10 shows a graph of the viscosity of Arg-Glu containing formulations vs Arg-HCl formulations

FIG. 11 shows a graph showing the viscosity of formulations comprising 150 mM Arg-HCl+ additional excipients

FIG. 12 shows a graph comparing the viscosity of 150 mM Arg-HCl formulations at different pH

FIG. 13 shows a graph comparing the viscosity of 20 mM vs 50 mM His buffer formulations FIG. 14 shows a graph comparing the viscosity of Formulations 16 to 30

FIG. 15 shows a series of graphs of viscosity measurements for Formulations 31 and 32 in comparison with Formulations 3 and 16

FIG. 16 show a series of graphs of the viscosity measurements for Formulations 16 to 30 (Formulation 40=200 mg/ml, all other formulations=175 mg/ml)

FIG. 17 shows a comparison of viscosity measurements for Formulations 33 to 45

FIG. 18 shows a graph of viscosity measurements for 175 mg/ml eblasakimab formulations FIG. 19 shows a graph of viscosity measurements for 150 mg/ml eblasakimab formulations FIG. 20 shows a graph of the stability test 3-month results for viscosity FIG. 21 shows a graph of the stability test 3-month results for osmolality FIG. 22 shows a graph of the stability test 3-month results for protein concentration (soloVPE). Order from left to right for each formulation: t0; t1m, 05C; t1m, 25C; t1m, 40C; t3m, 05C; t3m, 25C; and t3m, 40C

FIG. 23 shows a graph of the stability test 3-month results for pH. Order from left to right for each formulation: t0; t1m, 05C; t1m, 25C; t1m, 40C; t3m, 05C; t3m, 25C; and t3m, 40C

FIG. 24 shows a table of the stability test 3-month results based on visual inspection of the formulations

FIG. 25 shows a graph of the stability test 3-month results for turbidity. Order from left to right for each formulation: t0; t1m, 05C; t1m, 25C; t1m, 40C; t3m, 05C; t3m, 25C; and t3m, 40C

FIG. 26 shows a table of the stability test 3-month results for colour. Order from left to right for each formulation: t0; t1m, 05C; t1m, 25C; t1m, 40C; t3m, 05C; t3m, 25C; and t3m, 40C

FIG. 27A shows a graph of the stability test 3-month results for subvisible particle content using micro-flow imaging (MFI), ≥2 μm. Order from left to right for each formulation: t0; t1m, 05C; t1m, 25C; t1m, 40C; t3m, 05C; t3m, 25C; and t3m, 40C

FIG. 27B shows a graph of the stability test 3-month results for subvisible particle content (MFI), ≥10 μm. Order from left to right for each formulation: t0; t1m, 05C; t1m, 25C; t1m, 40C; t3m, 05C; t3m, 25C; and t3m, 40C

FIG. 27C shows a graph of the stability test 3-month results for subvisible particle content (MFI), ≥25 μm. Order from left to right for each formulation: t0; t1m, 05C; t1m, 25C; t1m, 40C; t3m, 05C; t3m, 25C; and t3m, 40C

FIG. 28 shows a table of the stability test 3-month results for protein mass recovery (HP-SEC). Order from left to right for each formulation: t0; t1m, 05C; t1m, 25C; t1m, 40C; t3m, 05C; t3m, 25C; and t3m, 40C

FIG. 29A shows a graph of the stability test 3-month results for aggregate % content (HP-SEC). Order from left to right for each formulation: t0; t1m, 05C; t1m, 25C; t1m, 40C; t3m, 05C; t3m, 25C; and t3m, 40C

FIG. 29B shows a graph of the stability test 3-month results for monomer % content (HP-SEC). Order from left to right for each formulation: t0; t1m, 05C; t1m, 25C; t1m, 40C; t3m, 05C; t3m, 25C; and t3m, 40C

FIG. 29C shows a graph of the stability test 3-month results for fragment % content (HP-SEC). Order from left to right for each formulation: t0; t1m, 05C; t1m, 25C; t1m, 40C; t3m, 05C; t3m, 25C; and t3m, 40C

FIG. 29D shows a summary table a comparison of aggregate, monomer and fragment % content with the 100 mg/ml eblasakimab formulation (HP-SEC)

FIG. 30A shows a graph of the stability test 3-month results for non-reducing aggregate % content (cGE). Order from left to right for each formulation: t0; t1m, 05C; t1m, 25C; t1m, 40C; t3m, 05C; t3m, 25C; and t3m, 40C

FIG. 30B shows a graph of the stability test 3-month results for non-reducing monomer % content (cGE). Order from left to right for each formulation: t0; t1m, 05C; t1m, 25C; t1m, 40C; t3m, 05C; t3m, 25C; and t3m, 40C

FIG. 30C shows a graph of the stability test 3-month results for non-reducing intact protein % content (cGE). Order from left to right for each formulation: t0; t1m, 05C; t1m, 25C; t1m, 40C; t3m, 05C; t3m, 25C; and t3m, 40C

FIG. 30D shows a summary table a comparison of intact protein % content with the 100 mg/ml eblasakimab formulation

FIG. 31A shows a graph of the stability test 3-month results for reducing non-glycosylated heavy chain (NGHC) % content (cGE). Order from left to right for each formulation: t0; t1m, 05C; t1m, 25C; t1m, 40C; t3m, 05C; t3m, 25C; and t3m, 40C

FIG. 31B shows a graph of the stability test 3-month results for reducing heavy chain (HC) % content (cGE). Order from left to right for each formulation: t0; t1m, 05C; t1m, 25C; t1m, 40C; t3m, 05C; t3m, 25C; and t3m, 40C

FIG. 31C shows a graph of the stability test 3-month results for reducing light chain (LC) % content (cGE). Order from left to right for each formulation: t0; t1m, 05C; t1m, 25C; t1m, 40C; t3m, 05C; t3m, 25C; and t3m, 40C

FIG. 31D shows a graph of the stability test 3-month results for reducing LC+HC % content (cGE). Order from left to right for each formulation: t0; t1m, 05C; t1m, 25C; t1m, 40C; t3m, 05C; t3m, 25C; and t3m, 40C

FIG. 31E shows a graph of the stability test 3-month results for reducing impurity % content (cGE). Order from left to right for each formulation: t0; t1m, 05C; t1m, 25C; t1m, 40C; t3m, 05C; t3m, 25C; and t3m, 40C

FIG. 31F shows a summary table a comparison of reducing LC+HC % and NGHC % content with the 100 mg/ml eblasakimab formulation (cGE)

FIG. 32A shows a graph of the stability test 3-month results for acidic peak % content (IEX)

FIG. 32B shows a graph of the stability test 3-month results for natural peak % content (IEX)

FIG. 32C shows a graph of the stability test 3-month results for basic peak % content (IEX)

FIG. 32D shows a summary table a comparison of acidic, neutral and basic peak % content with the 100 mg/ml ASLAN004 formulation (IEX)

### ABBREVIATIONS

BDP Bulk Drug Product iRS Interim Reference Standard
CV Column Volume 2-AB2-aminobenzamide
HCCF Harvested Cell Culture Fluid PS-20 Polysorbate 20
QSFF Q-Sepharose Fast Flow UF/DF Ultra-filtration/Dia-filtration
IEC IsoElectric Chromatography TFF Tangential Flow Filtration
SEC Size Exclusion Chromatography MFI Micro-flow imaging

### EXAMPLES

### Example 1—Step 1 (Primary) Screen: Formulations 1 to 15

15 different formulations were initially produced:

TABLE 1
Composition of Formulations 1 to 15

| | | | | | | | | | Theoretical |
|---|---|---|---|---|---|---|---|---|---|
| | | | | | | | | | osmolality |
| No. | API | pH | Buffer | Excipient 1 | Excipient 2 | | Excipient 3 | Surfactant | [mOsmol/kg] |
| 1 | 200 | 6.5 | 20 mM | 100 mM | 180 mM | Sucrose | | 0.02% | 405 |
| | mg/ml | | His | Arg-HCl | | | | PS20 | |
| 2 | | 6.5 | 20 mM | 125 mM | 0 mM | NaCl | | | 275 |
| | | | His | Arg-HCl | | | | | |
| 3 | | 6.5 | 20 mM | 150 mM | 0 mM | NaCl | | | 325 |
| | | | His | Arg-HCl | | | | | |
| 4 | | 6.5 | 20 mM | 175 mM | 50 mM | NaCl | | | 475 |
| | | | His | Arg-HCl | | | | | |
| 5 | | 6.5 | 20 mM | 200 mM | 0 mM | NaCl | | | 425 |
| | | | His | Arg-HCl | | | | | |
| 6 | | 6.5 | 20 mM | 150 mM | 50 mM | NaCl | | | 425 |
| | | | His | Arg-HCl | | | | | |
| 7 | | 6.5 | 20 mM | 100 mM | 100 mM | NaCl | | | 425 |
| | | | His | Arg-HCl | | | | | |
| 8 | | 6.5 | 20 mM | 150 mM | 75 mM | Serine/ | | | 475 |
| | | | His | Arg-HCl | | Threonine | | | |
| 9 | | 6.5 | 20 mM | 150 mM | 75 mM | Serine/ | 50 mM | | 525 |
| | | | His | Arg-HCl | | Threonine | Glycine | | |
| 10 | | 6.5 | 20 mM | 300 mM | | | | | 325 |
| | | | His | Serine | | | | | |
| 11 | | 6.5 | 20 mM | 150 mM | 150 mM | Glycine | | | 475 |
| | | | His | Arg-HCl | | | | | |
| 12 | | 6.5 | 20 mM | 150 mM | 150 mM | Proline | | | 475 |
| | | | His | Arg-HCl | | | | | |
| 13 | | 6.5 | 20 mM | 150 mM | 150 mM | Threonine | | | 475 |
| | | | His | Arg-HCl | | | | | |
| 14 | | 6.5 | 20 mM | 150 mM | 100 mM | Serine | | | 425 |
| | | | His | Arg-HCl | | | | | |
| 15 | | 6.5 | 20 mM | 200 mM | | | | | 425 |
| | | | His | Arg-Glu | | | | | |

Formulations 1-7: Arg-HCl/NaCl screening
Formulations 8-13: Ser, Thr, Gly, Pro screening
Formulations 14-15: Different arginine combinations

The formulations were produced as follows:
1. Eblasakimab BDP samples were concentrated 2 times to ˜7.5 ml and diluted with 7.5 ml formulation buffer.
2. Formulation homogenized using positive displacement pipette.
3. Steps 1 and 2 were repeated 8 times.
4. After the 8th dilution step, each formulation was concentrated to yield the target concentration of 150 mg/ml, 175 mg/ml or 200 mg/ml.
5. Each formulation underwent sterile filtration.
6. Polysorbate-20 was added to achieve a target concentration of 0.02% (w/w).
7. Protein concentration (Solo-VPE), osmolarity and pH was measured for each formulation.
8. Vials were filed with the formulations (1×1 ml)
9. Any residual samples were stored at 2-8° (CW 18 viscosity/HP-SEC)

### Viscosity Studies

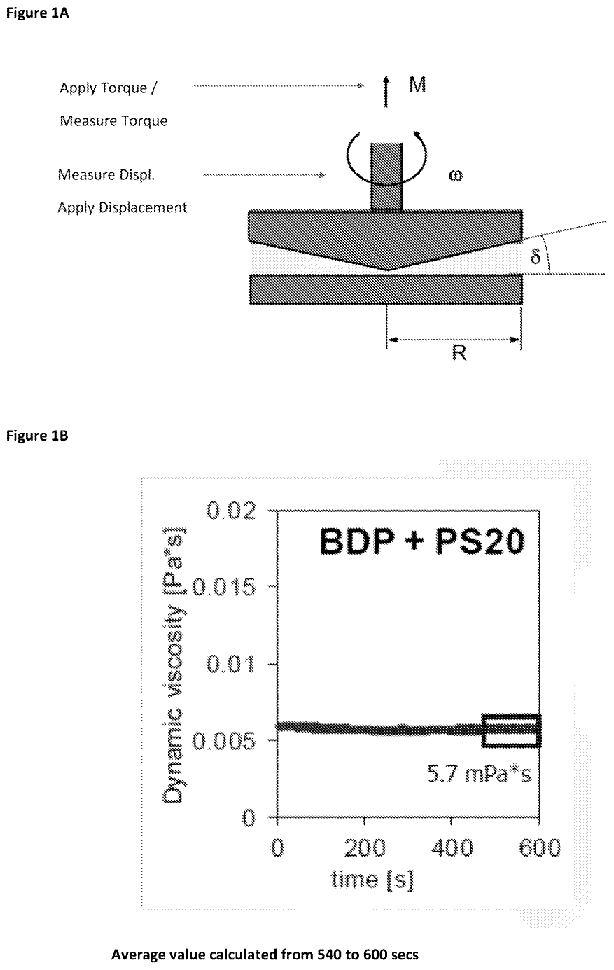

The viscosity of each formulation was tested using a Kinexus ultra and rheometer. See FIG. 1A. Measurements were carried out at 20° at 1000 rpm/sec. The cone plates used were:
40 mm, 1° angle, 24 μm gap, for samples<10 cP (mPas)·
20 mm, 1° angle, 24 μm gap, for samples>10 cP (mPas)

The average dynamic viscosity values were calculated from the reading between 540 to 600 sec. The eblasakimab samples are likely to behave as non-Newtonian fluids and the shear rate applied is constant. Thus, in the context of the present disclosure, the dynamic viscosity and shear viscosity are taken to be the same and these terms are used interchangeably in the Examples.

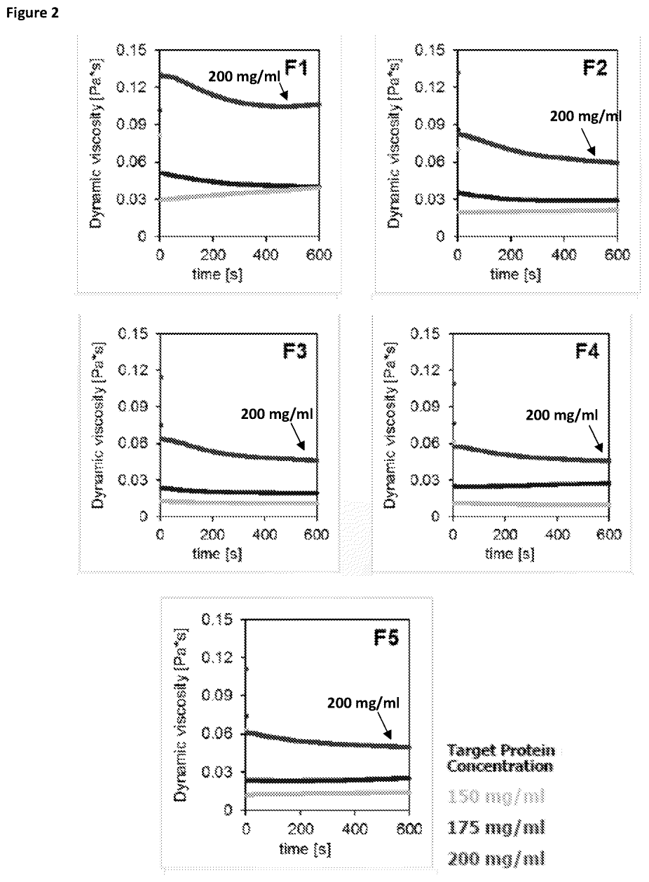

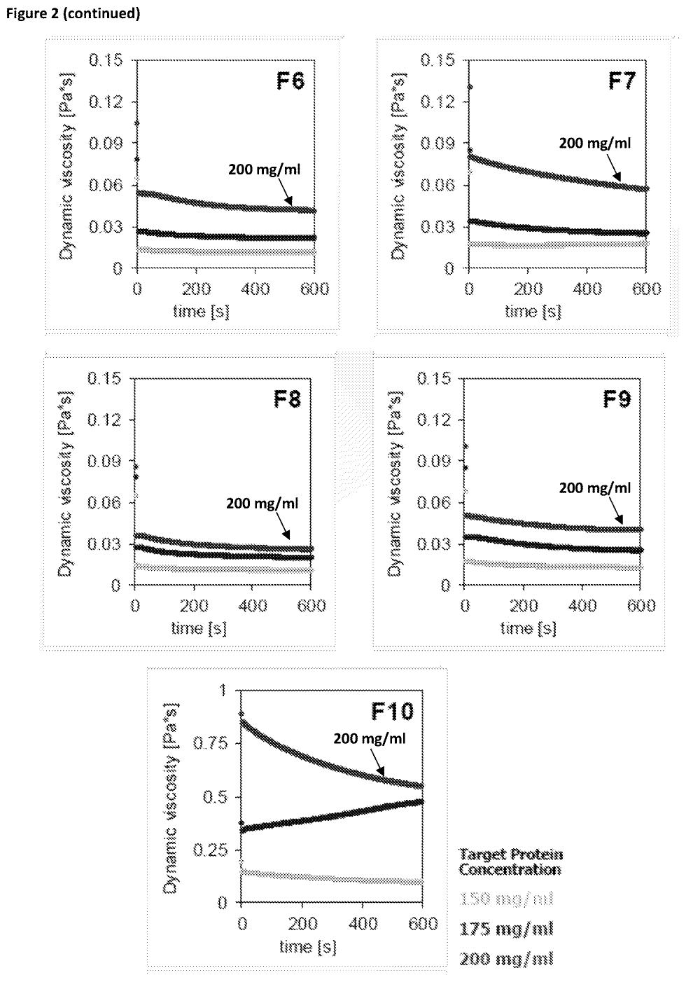

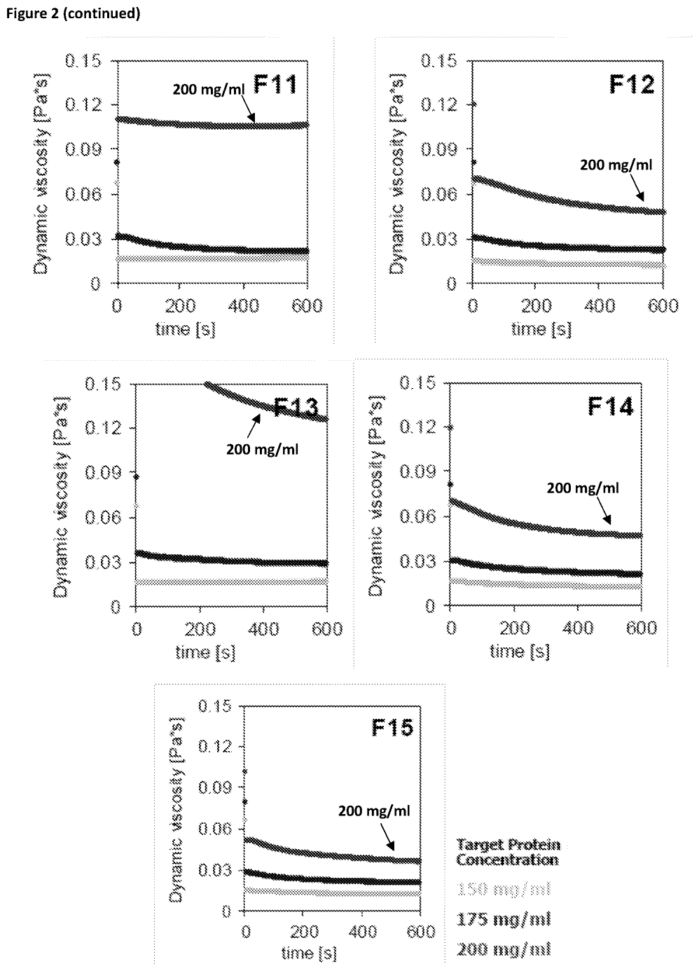

FIG. 1B shows the viscosity measurements for the eblasakimab BDP with polysorbate 20 added. The results of the viscosity measurements for Formulations 1 to 15 are shown in FIG. 2.

Table 2 below shows the formulations ranked by viscosity at different target concentration ranges.

TABLE 2
Viscosity measurements for Formulations 1 to 15 for ASLAN004 150 mg/ml, 175 mg/ml and 200 mg/ml

| target 150 mg/ml | | | target 175 mg/ml | | | target 200 mg/ml | | |
|---|---|---|---|---|---|---|---|---|
| Protein | | Dynamic | Protein | | Dynamic | Protein | | Dynamic |
| Concentration | | viscosity | Concentration | | viscosity | Concentration | | viscosity |
| [mg/ml] | Formulation | [mPa*s] | [mg/ml] | Formulation | [mPa*s] | [mg/ml] | Formulation | [mPa*s] |
| 159 | F4 | 9.8 | 172 | F3 | 19.3 | 185 | F8 | 26.1 |
| 148 | F8 | 10.9 | 176 | F8 | 20.2 | 197 | F15 | 36.6 |
| 154 | F3 | 11.0 | 174 | F15 | 20.9 | 189 | F9 | 40.7 |
| 154 | F6 | 11.3 | 175 | F14 | 21.2 | 202 | F6 | 41.8 |
| 155 | F12 | 12.0 | 179 | F11 | 21.4 | 214 | F4 | 45.9 |
| 152 | F15 | 12.4 | 177 | F6 | 21.6 | 211 | F3 | 46.4 |
| 151 | F14 | 12.5 | 182 | F12 | 22.4 | 204 | F14 | 47.0 |
| 151 | F9 | 12.9 | 177 | F5 | 25.1 | 205 | F12 | 48.1 |
| 152 | F5 | 14.0 | 169 | F7 | 25.2 | 209 | F5 | 49.7 |
| 151 | F13 | 16.7 | 171 | F9 | 25.2 | 203 | F7 | 57.7 |
| 147 | F11 | 16.9 | 175 | F4 | 27.2 | 204 | F0 | 59.6 |
| 149 | F7 | 17.6 | 178 | F2 | 28.9 | 204 | F1 | 105.6 |
| 155 | F2 | 21.3 | 179 | F13 | 29.0 | 215 | F11 | 106.3 |
| 160 | F1 | 38.7 | 178 | F1 | 40.2 | 219 | F13 | 127.1 |
| 155 | F10 | 98.2 | 167 | F10 | 470.5 | 178 | F10 | 555.0 |

The results indicate that most of the 150 mg/ml formulations and several 175 mg/ml formulations are close to the 20-25 cP viscosity target.

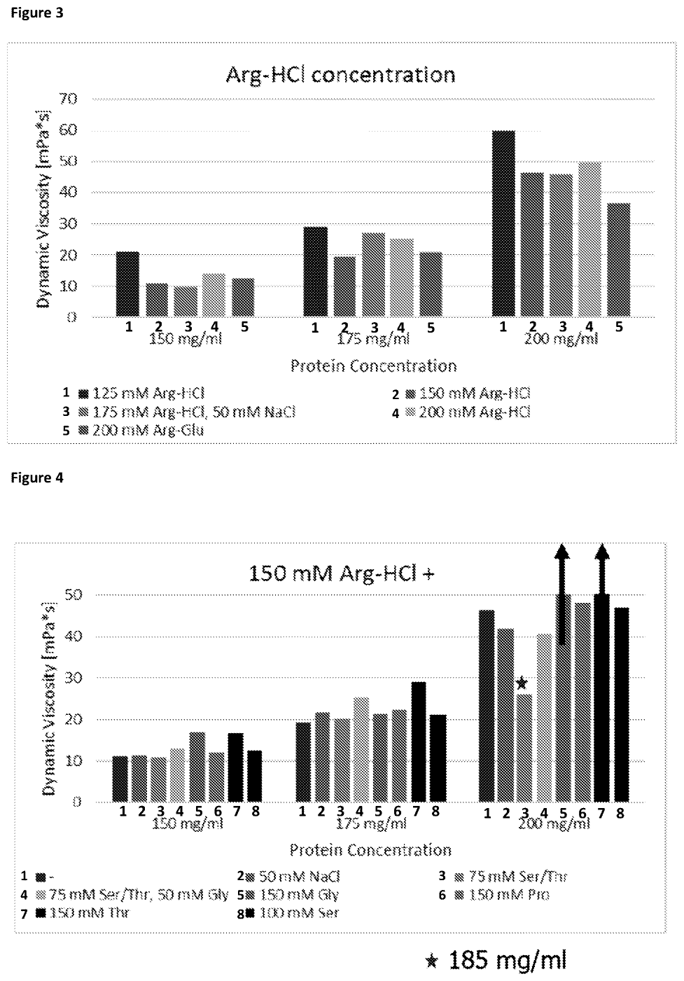

FIG. 3 shows the relationship between Arg-HCl concentration and viscosity. The results suggest that minimal viscosity is reached with 150 mM Arg-HCl and that higher Arg-HCl concentration does not further reduce viscosity. However, this was investigated further below.

FIG. 4 shows the impact of the different excipients on viscosity. The results indicate that Arg-Glu reduces viscosity similarly to Arg-HCl and that adding an additional excipient to 150 mM Arg-HCl did not further reduce viscosity. This was investigated further below.

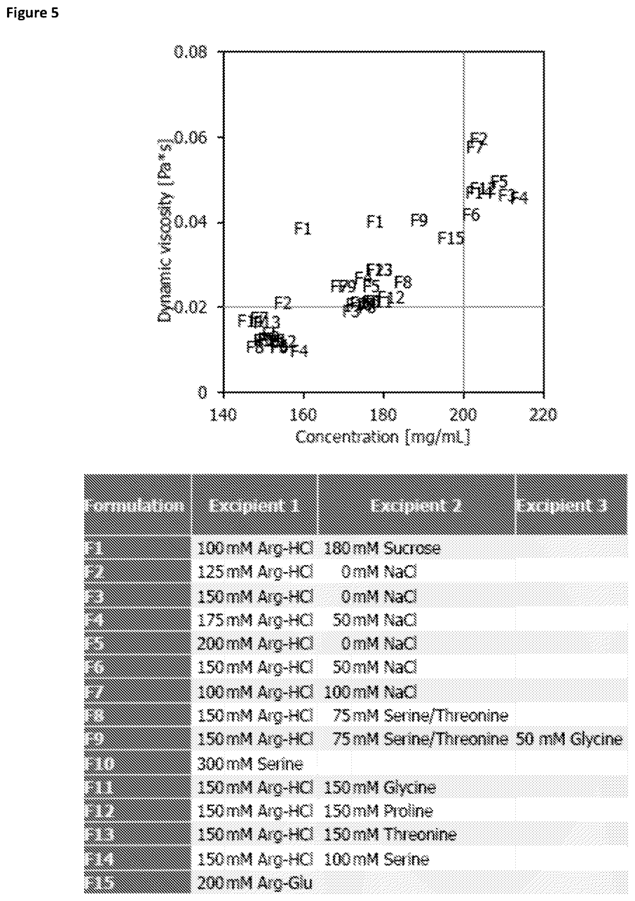

FIG. 5 shows the correlation between viscosity and ASLAN004 concentration.

The results suggest that Formulation 3 had the best overall viscosity across the 3 antibody concentrations.

The data herein is not the full data set but is exemplary data to illustrate the trends.

### Example 2—Step 2 (Secondary) Screen: Formulations 16 to 30

Using Formulation 3 as a starting point, a further 15 formulations were produced in an attempt to develop a suitable 200 mg/ml ASLAN004 formulation. See Table 3 below.

TABLE 3
Composition of Formulations 16 to 30

| | | | | | | | | Theoretical |
|---|---|---|---|---|---|---|---|---|
| | | | | | | | | osmolality |
| No. | API | pH | Buffer | Excipient 1 | Excipient 2 | | Surfactant | [mOsmol/kg] |
| F16 | 200 mg/ml | 6.5 | 20 mM His | 150 mM Arg-HCl | | | 0.02% PS20 | 325 |
| F17 | | 6.5 | 20 mM His | 150 mM Arg-HCl | 50 mM | Lys-HCl | | 425 |
| F18 | | 6.5 | 20 mM His | 150 mM Arg-HCl | 50 mM | Leucine | | 375 |
| F19 | | 6.5 | 20 mM His | 150 mM Arg-HCl | 50 mM | Valine | | 375 |
| F20 | | 6.5 | 20 mM His | 150 mM Arg-HCl | 50 mM | Phenylalanine | | 375 |
| F21 | | 6.5 | 20 mM His | 150 mM Arg-HCl | 50 mM | CaCl₂ | | 425 |
| F22 | | 6.5 | 20 mM His | 150 mM Arg-Glu | | | | 325 |
| F23 | | 6.5 | 50 mM His | 150 mM Arg-HCl | | | | 364 |
| F24 | | 6.0 | 20 mM His | 150 mM Arg-HCl | | | | 331 |
| F25 | | 6.5 | 20 mM His | 175 mM Arg-HCl | | | | 375 |
| F26 | | 6.5 | 50 mM His | 175 mM Arg-HCl | | | | 414 |
| F27 | | 6.5 | 20 mM His | 175 mM Arg-Glu | | | | 378 |
| F28 | | 6.5 | 20 mM His | 250 mM Arg-Glu | | | | 525 |
| F29 | | 6.5 | 20 mM His | 250 mM Arg-HCl | | | | 525 |
| F30 | | 7.0 | 20 mM His | 157 mM Arg-HCl | | | | 325 |

Formulations 16-21: Additional excipient screening
Formulations 22-30: Different pH, His buffer concentration and Arginine concentration

The formulations were prepared as follows:
1. 10 ml eblasakimab BDP+5 ml formulation buffer was transferred into an Amicon-15 unit (MWCO 100 kDa)
2. Formulation concentrated 2 times to ˜7.5 ml and diluted with 7.5 ml formulation buffer.
3. Formulation homogenized using positive displacement pipette.
4. Steps 2 and 3 were repeated 4 times.
5. After the 5^{th} dilution step, each formulation was concentrated to yield the target concentration of 150 mg/ml, 175 mg/ml or 200 mg/ml.
6. Each formulation underwent sterile filtration (0.8/0.2 μm PES).
7. Poly-sorbate-20 was added to achieve a target concentration of 0.02% (w/w).
8. Protein concentration (Solo-VPE), osmolarity and pH was measured for each formulation.
9. Any residual samples were stored at 2-8° (for viscosity determination)

### Viscosity Studies

The viscosity of each formulation was tested using the same method described in Example 1 above.

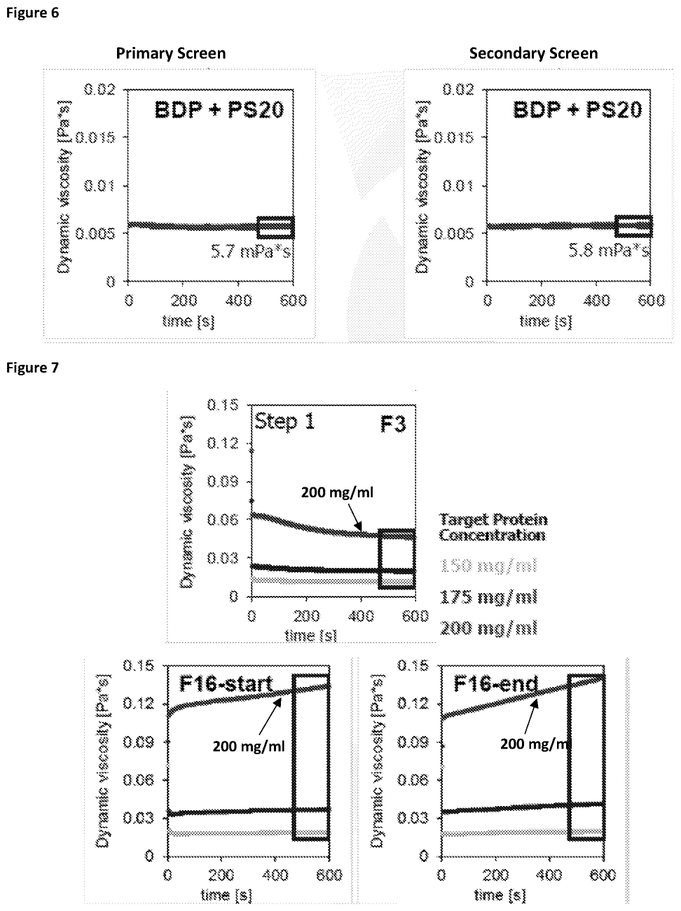

FIG. 6 shows a comparison between the viscosity measurement for the eblasakimab BDP from the primary (Example 1) and secondary screen (Example 2). As can be seen the viscosity values are very similar, suggesting that ASLAN004 BDP used in both screens was comparable.

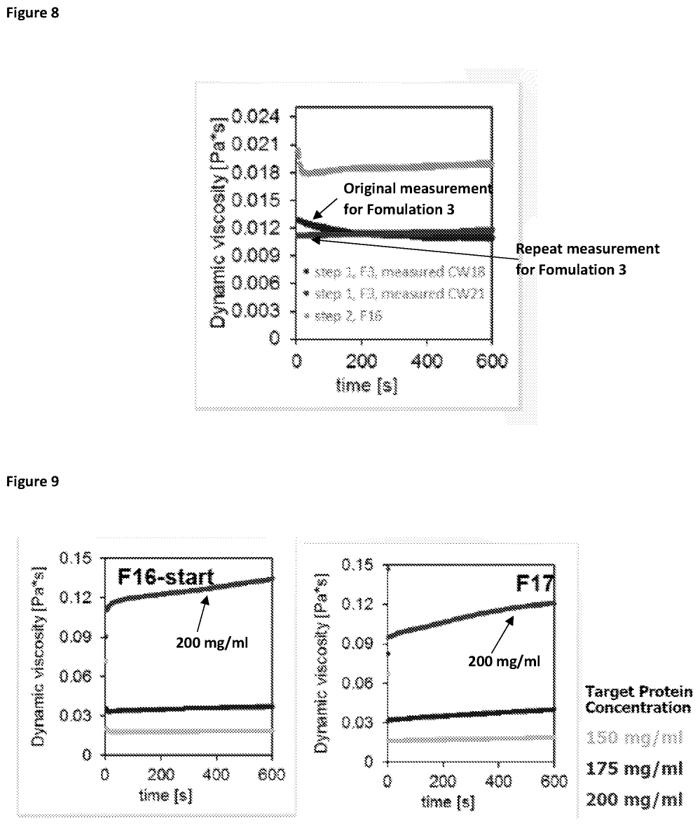

FIG. 7 shows a comparison between Formulation 3 and Formulation 16. Note FIG. 8 indicates that the viscosity reading for Formulation 3 was reproducible. These results demonstrated that the measurement method was reliable and that the viscosity readings were useful for assessing the formulations.

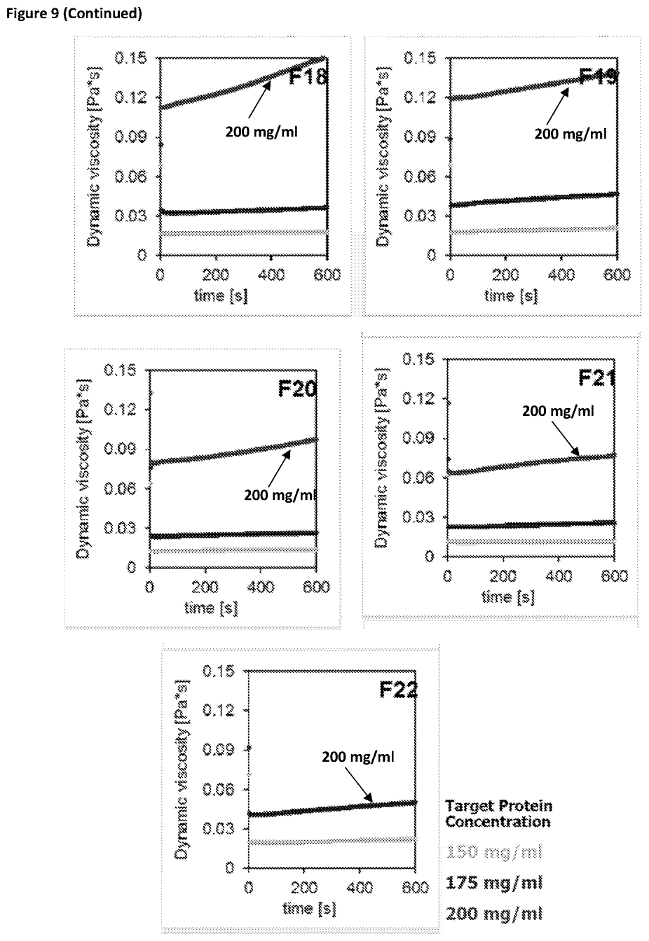

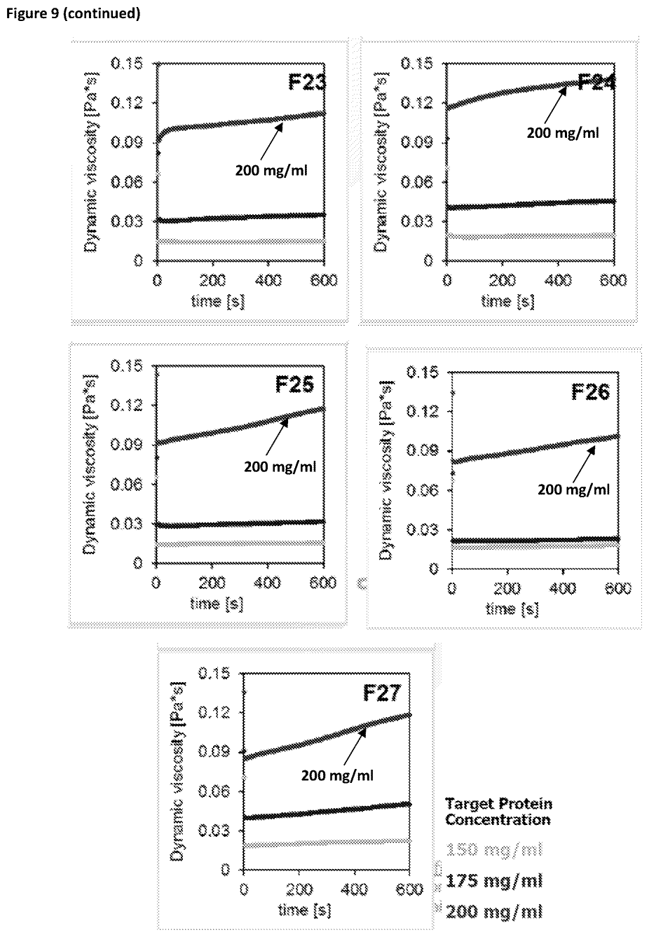

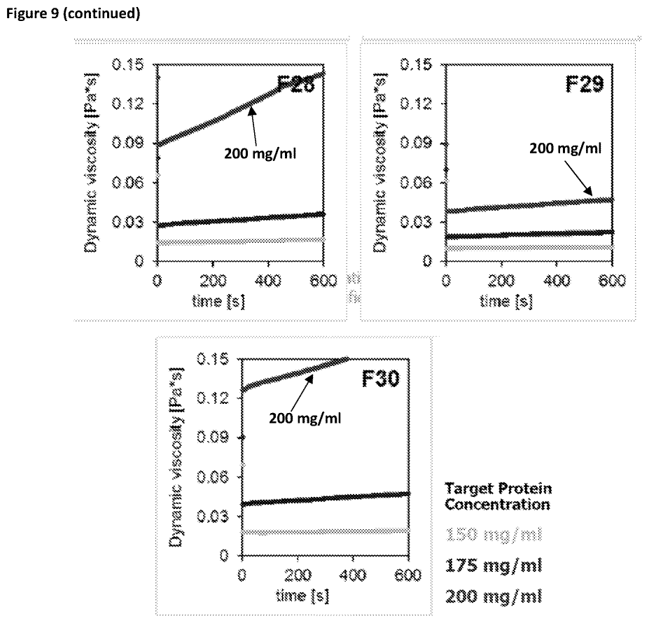

The results of the viscosity measurements for Formulations 16 to 30 are shown in FIG. 9. Table 4 below shows the formulations ranked by viscosity at different target concentration ranges.

TABLE 4
Viscosity measurements for Formulations 16 to 30
for ASLAN004 150 mg/ml, 175 mg/ml and 200 mg/ml

| target 150 mg/ml | | | Target 175 mg/ml | | | Target 200 mg/ml | | |
|---|---|---|---|---|---|---|---|---|
| | Protein | Dynamic | | Protein | Dynamic | | Protein | Dynamic |
| | conc. | viscosity | | conc. | viscosity | | conc. | viscosity |
| Formulation | (mg/ml) | (mPa*s) | Formulation | (mg/ml) | (mPa*s) | Formulation | (mg/ml) | (mPa*s) |
| F29 | 151 | 11 | F29 | 172 | 22 | F29 | 203 | 47 |
| F21 | 154 | 12 | F26 | 182 | 23 | F21 | 211 | 76 |
| F20 | 153 | 14 | F21 | 178 | 26 | F20 | 217 | 96 |
| F23 | 153 | 15 | F20 | 179 | 26 | F26 | 204 | 100 |
| F25 | 152 | 16 | F25 | 185 | 32 | F23 | 212 | 111 |
| F28 | 154 | 17 | F23 | 182 | 35 | F25 | 216 | 116 |
| F18 | 157 | 18 | F28 | 174 | 36 | F27 | 200 | 117 |
| F26 | 158 | 18 | F18 | 183 | 36 | F17 | 210 | 120 |
| F17 | 155 | 19 | F16-start | 176 | 37 | F16-start | 212 | 133 |
| F16-start | 155 | 19 | F17 | 179 | 40 | F-24 | 211 | 138 |
| F30 | 155 | 19 | F16-end | 178 | 41 | F19 | 214 | 138 |
| F24 | 153 | 20 | F24 | 181 | 46 | F16-end | 214 | 139 |
| F16-end | 155 | 20 | F19 | 178 | 46 | F28 | 211 | 141 |
| F19 | 153 | 21 | F30 | 178 | 47 | F18 | 217 | 149 |
| F22 | 155 | 22 | F22 | 179 | 50 | F30 | 214 | 162 |
| F27 | 149 | 22 | F27 | 170 | 50 | F22 | 218 | 229 |

The results indicate that all of 150 mg/ml formulations and several 175 mg/ml formulations were close to the 20 cP visocity target. Surprisingly many of the of the 200 mg/ml formulations had viscosity values that were significantly lower than the predicted viscosity. High concentrations of Arg-HCl (such as 250 Mm) significantly reduced viscosity at the 200 mg/ml formulation. 200 mg/ml formulations with high concentrations of Arg-HCl (such as 250 Mm) in combination with a salt such as CaCl₂) (in particular 50 Mm) and/or an amino acid such as phenylalanine (in particular 50-75 mM) may provided a viscosity of about 20 cP.

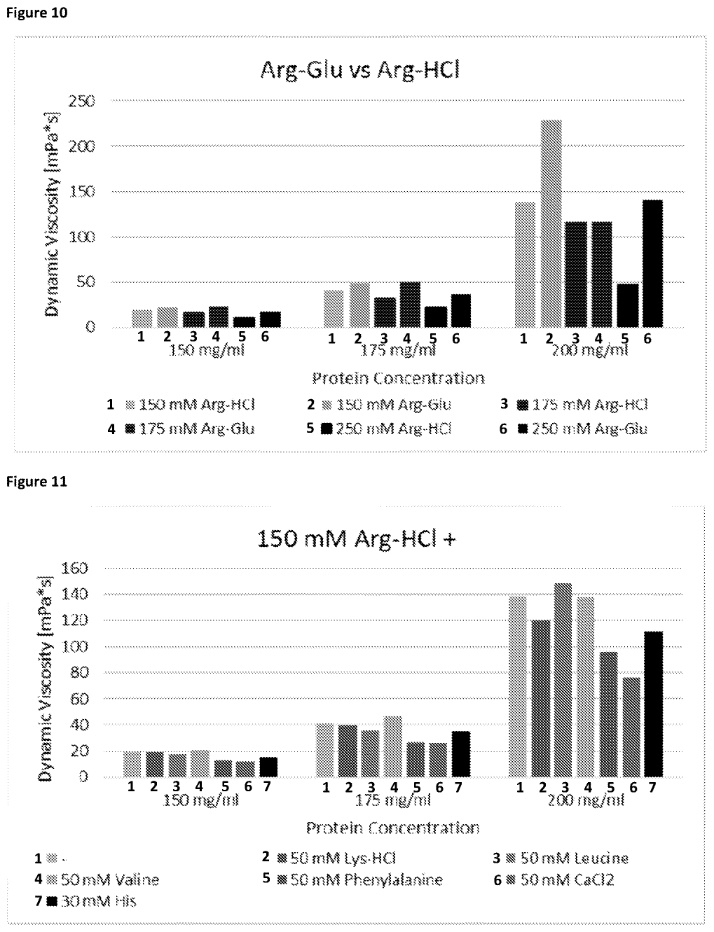

FIG. 10 shows a comparison between the viscosities of Arg-HCl and Arg-Glu formulations at various concentrations. The results suggest that there was a more pronounced reduction in viscosity for the Arg-HCl compared to the Arg-Glu formulations. In particular, increasing the Arg-HCl from 175 mM to 250 mM seemed to reduce the viscosity substantially.

FIG. 11 shows the impact of the different excipients on viscosity. The results indicate that the addition of Phenylalanine and CaCl₂) helps to reduce viscosity.

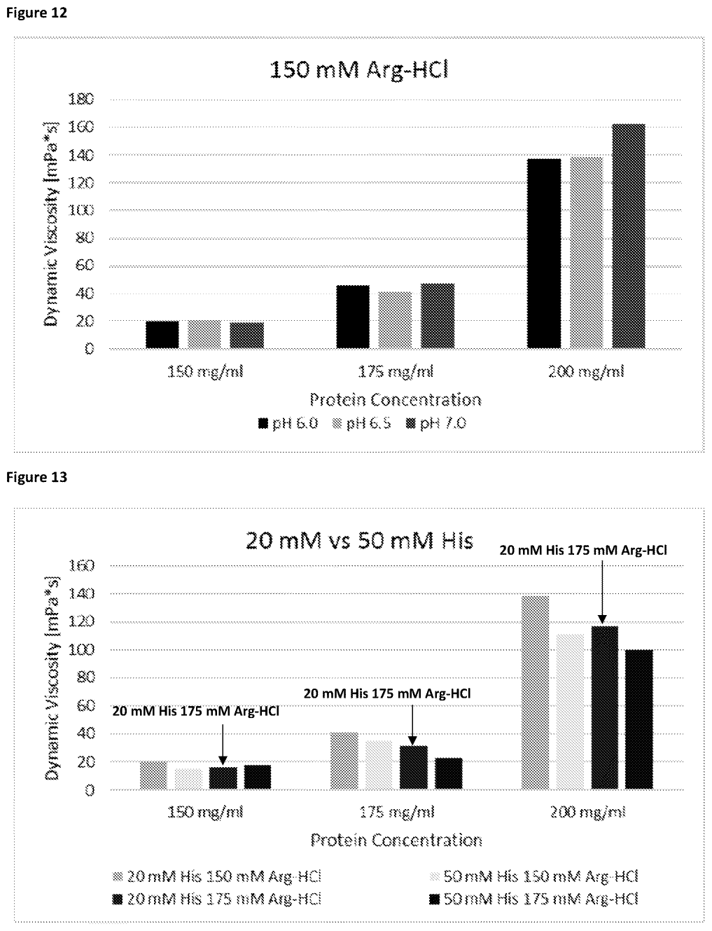

FIG. 12 shows the impact of pH on viscosity for 150 mM Arg-HCl formulations. The results demonstrate that increasing the pH from 6.5 to 7 or reducing it to 6.0 did not improve viscosity. Thus, it would seem that any pH between 6.0 to 7.0 is suitable.

FIG. 13 shows the results of an experiment to assess the impact of increasing His buffer concentration. The results suggest that increasing the histidine concentration from 20 to 50 mM for the 175 mg/ml and 200 mg/ml ASLAN004 formulations reduce the viscosity.

FIG. 14 shows the correlation between viscosity and ASLAN004 concentration.

The results suggest that Formulation 29 had the best overall viscosity across the 3 antibody concentrations.

### Example 3—3^{rd} Step Screen

The purpose of the 3^{rd} screen was to attempt to gain a better understanding of the viscosity differences observed between the 1^{st} and 2^{nd} step screens and to confirm the formulations' viscosity range for the formulation stress testing.

Based on the 1^{st} and 2^{nd} step screens, Formulations 3/16 and 29 had the best overall viscosities. A further 2 formulations were prepared based on these formulations:

| | | | | | | Theoretical | |
|---|---|---|---|---|---|---|---|
| | | | | | | osmolality | Nominal |
| No. | pH | Buffer | Excipient 1 | Excipient 2 | Surfactant | [mOsmol/kg] | same as |
| F31 | 6.5 | 20 mM His | 150 mM Arg-HCl | | 0.02% PS20 | 325 | F3, F16 |
| F32 | 6.5 | 20 mM His | 250 mM Arg-HCl | | | 525 | F29 |

The formulations were prepared as follows:
1. 20 ml ASLAN004 BDP (100 mg/ml) was diluted to 63 ml (62 mg/ml)
2. Buffer exchange performed with 8 dilution volumes (˜24 hour run time)
3. Formulations concentrated in tangential flow filtration (TFF) to 166 mg/ml
4. Formulations concentrated in an Amicon unit to 208 mg/ml
5. Each formulation underwent sterile filtration (0.8/0.2 μm PES)
6. Finally, polysorbate-20 was spiked in
7. For Formulation 32, 100 mM Arg-HCl was spiked in

### Viscosity Studies

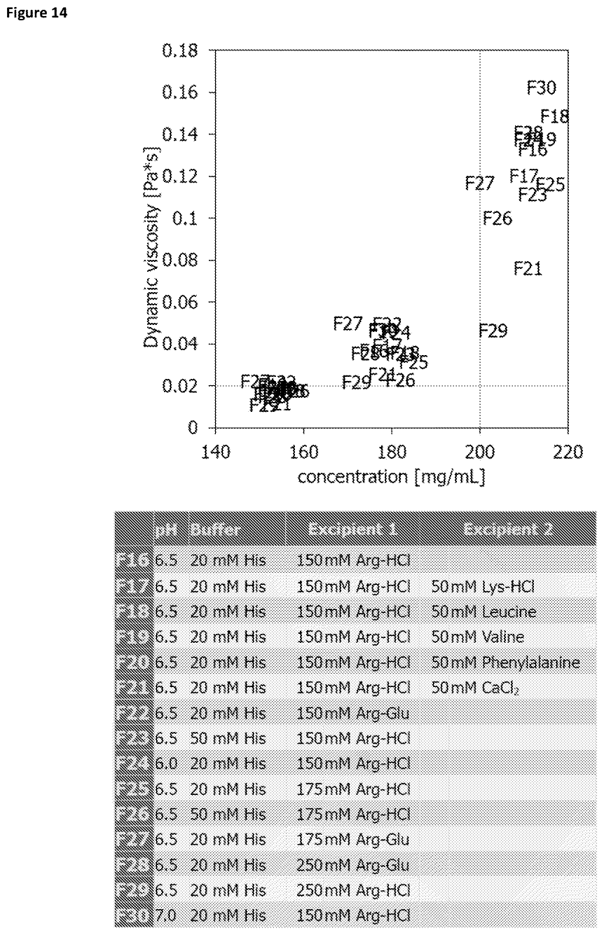

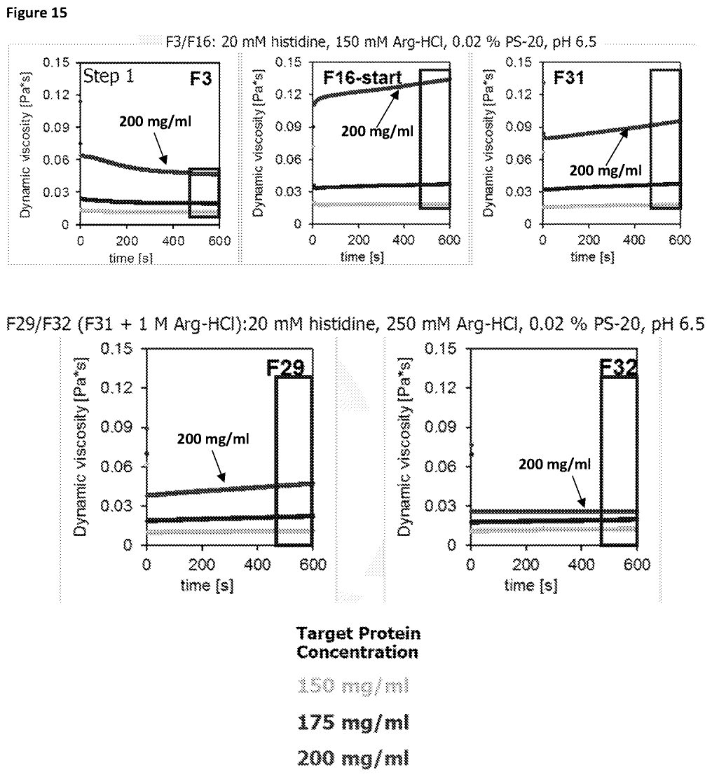

The viscosity of each formulation was tested using the same method described in Example 1 above. The results are shown in FIG. 15 and Table 5 below:

TABLE 5
Viscosity and actual protein concentration
measurements for Formulations 3, 16, 31, 29 and 32
Viscosity [mPa*s], protein concentration [mg/ml]

| Step 1, F3 | Step 2, F16 | Step 3, F31 |
|---|---|---|
| 11.0, 154 | 18.9, 155 | 18.2, 152 |
| 19.3, 172 | 37.0, 176 | 37.2, 175 |
| 46.4, 211 | 133.2, 212 | 94.6, 203 |

| Step 1, F29 | Step 2, F32 |
|---|---|
| 11.2, 151 | 12.5, 151 |
| 22.1, 172 | 19.7, 170 |
| 46.7, 203 | 26.0, 184 |

There was a good agreement between the viscosity of Formulations 29 and 32.

The results suggests that the increase in arginine concentration from 150 mM to 250 mM resulted in a substantial and consistent improvement to the viscosity 200 mg/ml ASLAN004.

### Example 4—4^{th} Step Screen

Using Formulation 29 as a starting point, a further 13 formulations were produced to further refine the 175 mg/ml formulations. See Table 6 below.

TABLE 6
Composition of Formulations 33 to 45

| | | | | | | | | Theoretical | |
|---|---|---|---|---|---|---|---|---|---|
| | | | His | Arg-HCl | Phe | CaCl₂ | | osmolality | Nominally |
| No. | API | pH | [mM] | [mM] | [mM] | [mM] | PS20 | [mOsmol/kg] | same as |
| F33 | 175 mg/ml | 6.5 | 20 | 250 | | | 0.02 | 525 | F29 |
| F34 | 175 mg/ml | 6.5 | 20 | 250 | 20 | 20 | (w/w)% | 605 | |
| F35 | 175 mg/ml | 6.5 | 20 | 250 | 50 | | | 575 | |
| F36 | 175 mg/ml | 6.5 | 20 | 200 | | 50 | | 575 | |
| F37 | 175 mg/ml | 6.5 | 20 | 275 | | | | 575 | |
| F38 | 175 mg/ml | 6.5 | 20 | 175 | 50 | 50 | | 575 | |
| F39 | 175 mg/ml | 6.5 | 20 | 250 | 75 | | | 600 | |
| F40 | 200 mg/ml | 6.5 | 20 | 275 | | | | 575 | |
| F41 | 175 mg/ml | 6.5 | 20 | 175 | 75 | 50 | | 600 | |
| F42 | 175 mg/ml | 6.5 | 50 | 175 | 63 | | | 477 | |
| F43 | 175 mg/ml | 6.5 | 20 | 175 | | 75 | | 600 | |
| F44 | 175 mg/ml | 6.5 | 50 | 175 | | | | 414 | F26 |
| F45 | 175 mg/ml | 6.5 | 50 | 200 | | | | 464 | |

The formulations were prepared using the same method described in the secondary screen (Example 2) above. Formulations 41 to 45 were prepared 2 weeks after formulations 33 to 40.

### Viscosity Studies

The viscosity of each formulation was tested using the same method described in Example 1 above.

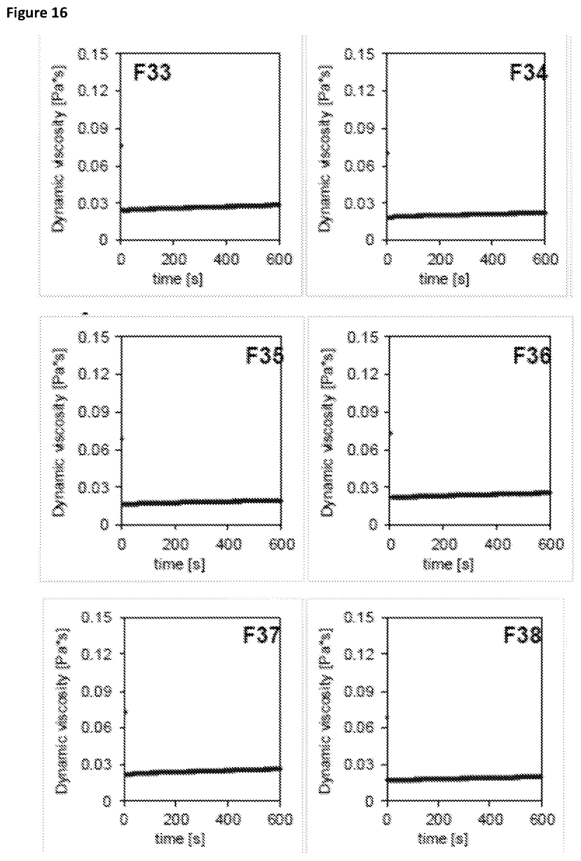

The results of the viscosity measurements for Formulations 33 to 45 are shown in FIG. 16. Table 7 below shows the formulations ranked bv viscosity.

TABLE 7
Viscosity measurements for Formulations 39
to 40 for ASLAN004 175 mg/ml and 200 mg/ml

| | Protein concentration | |
|---|---|---|
| Formulation | [mg/ml] | Shear viscosity (mPa*s) |
| F39 | 177 | 18.3 |
| F35 | 175 | 19.4 |
| F38 | 178 | 20.0 |
| F34 | 178 | 22.1 |
| F41 | 190 | 25.1 |
| F36 | 183 | 25.5 |
| F37 | 181 | 26.4 |
| F33 | 182 | 28.2 |
| F42 | 188 | 28.7 |
| F43 | 185 | 29.0 |
| F45 | 185 | 32.7 |
| F44 | 183 | 38.0 |
| F40 | 208 | 55.1 |

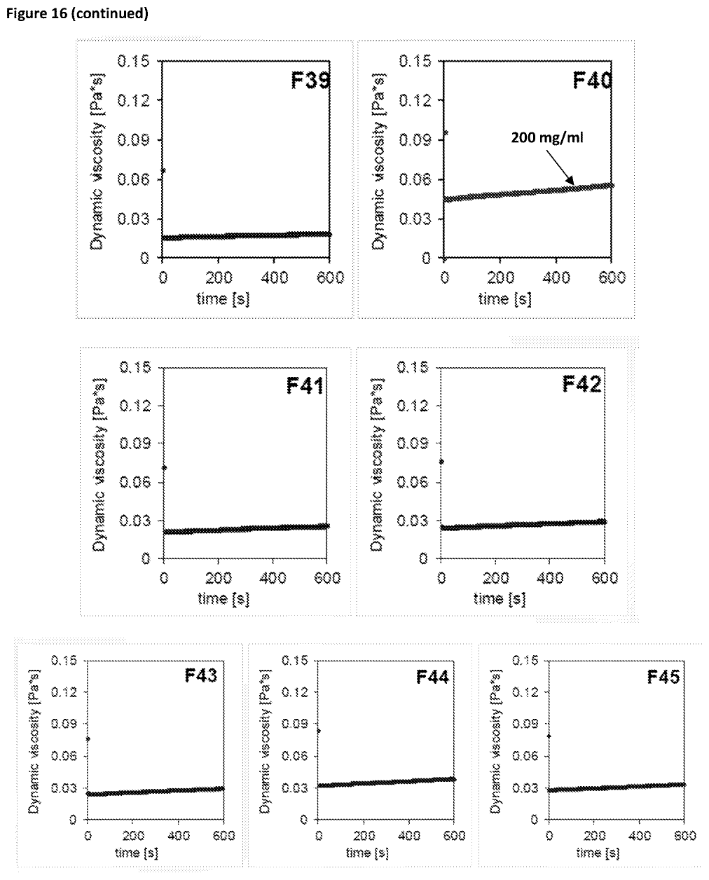

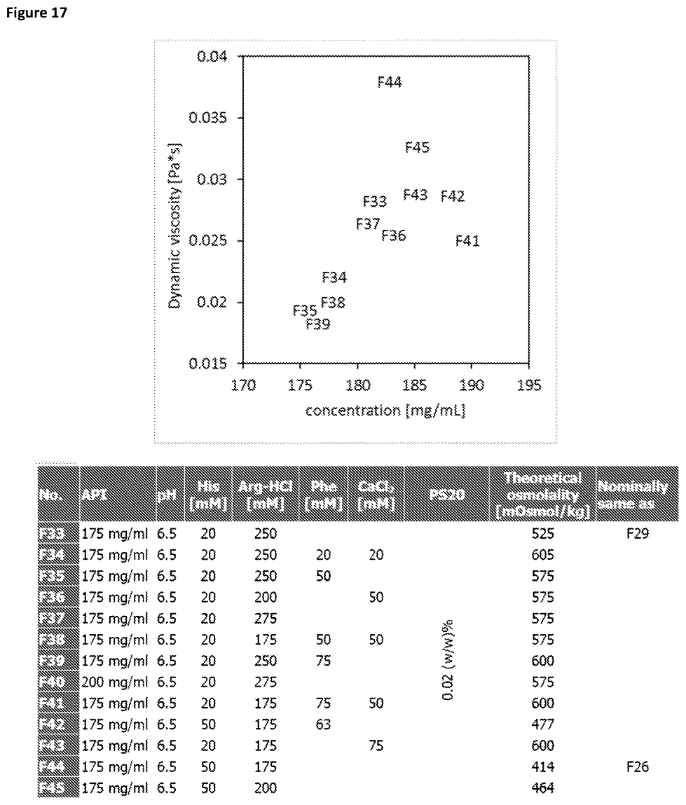

FIG. 17 shows the correlation between viscosity and eblasakimab concentration. The Phenylalanine formulations tended to cluster at lower viscosity values. This suggests that the addition of Phenylalanine to the formulations helps to reduce viscosity.

### Example 5—Formulation Selection

The viscosity results from the 2^{nd} to 4^{th} screens were compared to determine the best formulations to take forward to the stability studies.

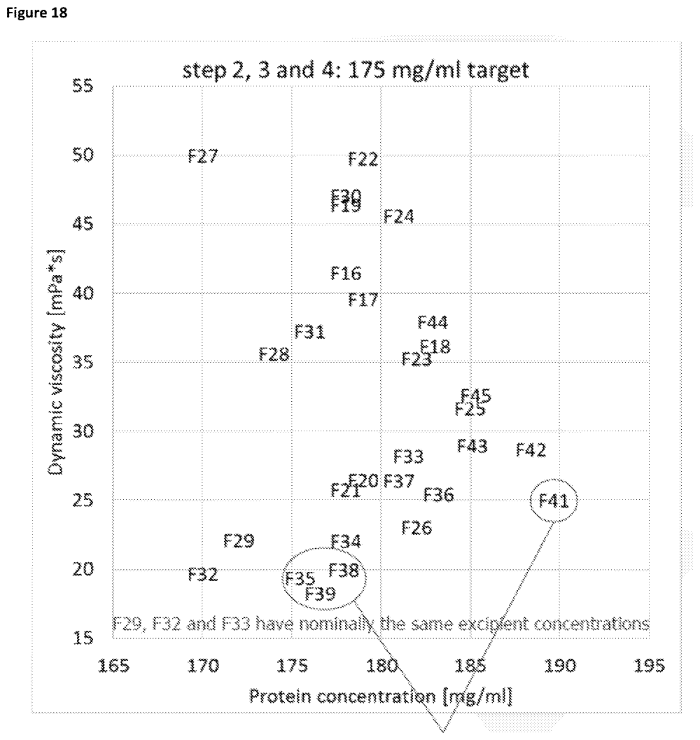

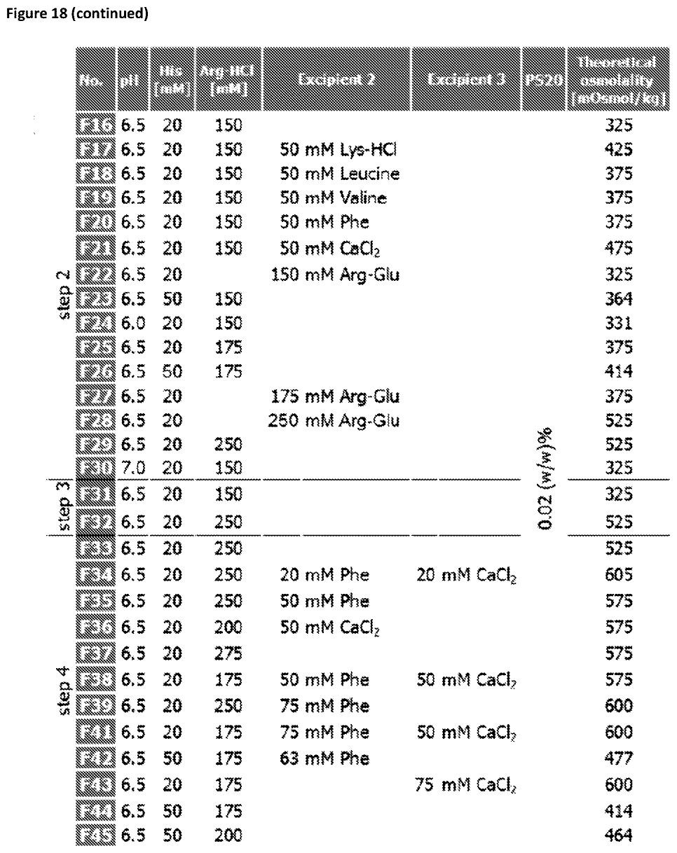

FIG. 18 shows an overview of the viscosity measurements for the 2^{nd}, 3^{rd} and 4^{th} screens for the 175 mg/ml eblasakimab formulations. The results suggest that the formulations tested in the 4th screen that comprised Arg-HCl and Phenylalanine had the lowest viscosities, i.e. Formulations 35, 38, 39 and 41.

FIG. 19 shows an overview of the viscosity measurements for the 2nd and 3rd screens for the 150 mg/ml eblasakimab formulations. The results suggest that Formulations 21 and 29 had the lowest viscosities.

Based on these results, the final list of formulations shown in Table 8 was selected.

TABLE 8
Final list of formulations selected

| | | | | | | | | Theoretical |
|---|---|---|---|---|---|---|---|---|
| | API | | His | Arg-HCl | Phe | Additional | | osmolality |
| No. | [mg/ml] | pH | [mM] | [mM] | [mM] | Excipients | PS20 | [mOsmol/kg] |
| F46 | 175 | 6.5 | 20 | 250 | 75 | | 0.02 | 600 |
| F47 | 175 | 6.5 | 20 | 175 | 80 | | (w/w)% | 455 |
| F48 | 150 | 6.5 | 20 | 175 | | 50 mM | | 525 |
| | | | | | | CaCl₂ | | |
| F49 | 175 | 6.5 | 20 | 100 | | 180 mM | | 405 |
| | | | | | | Sucrose | | |
| F50 | 175 | 6.5 | 20 | 175 | 75 | 50 mM | | 600 |
| | | | | | | CaCl₂ | | |
| F51 | 175 | 6.5 | 20 | 200 | 50 | | | 475 |

These formulations will be produced and progressed in stability/stress test studies.

### Example 6—Stability Tests-3 Month Interim Results

The 6 formulations in Table 8 were progressed in stability test studies whereby the formulations were stored at 2-8° C., 25° C. or 40° C. The 3-month interim results are shown in FIGS. 20 to 32.

FIG. 20 shows the viscosity results for the 6 formulations. The results suggest that the viscosities increased slightly after 3 months. In particular, the viscosities for the samples stored at 5° C. and 25° C. were comparable, whereas a more noticeable increase in viscosity was observed for the samples stored at 40° C.

FIG. 21 shows the osmolality results for the 6 formulations. The differences observed were within the expected method variability.

FIG. 22 shows the protein concentration results for the 6 formulations. There was no distinct change observed in the 3-month samples compared to the day 0 samples.

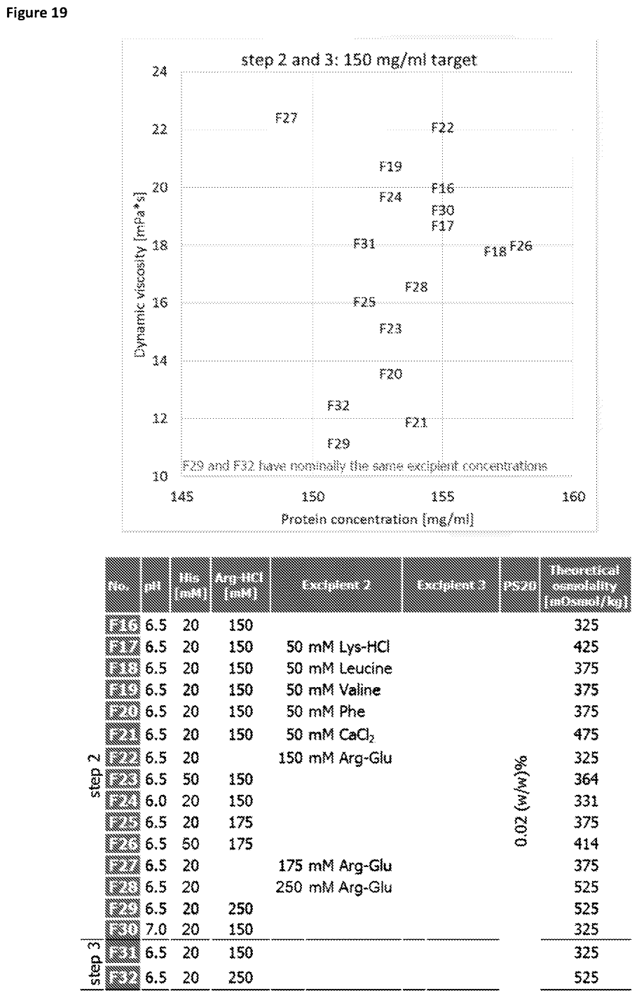

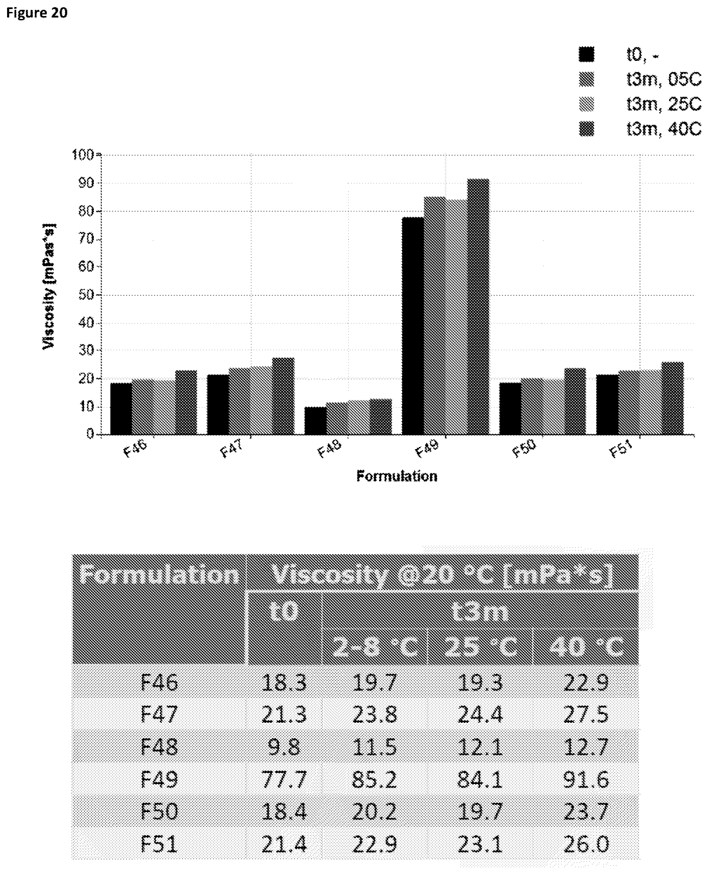

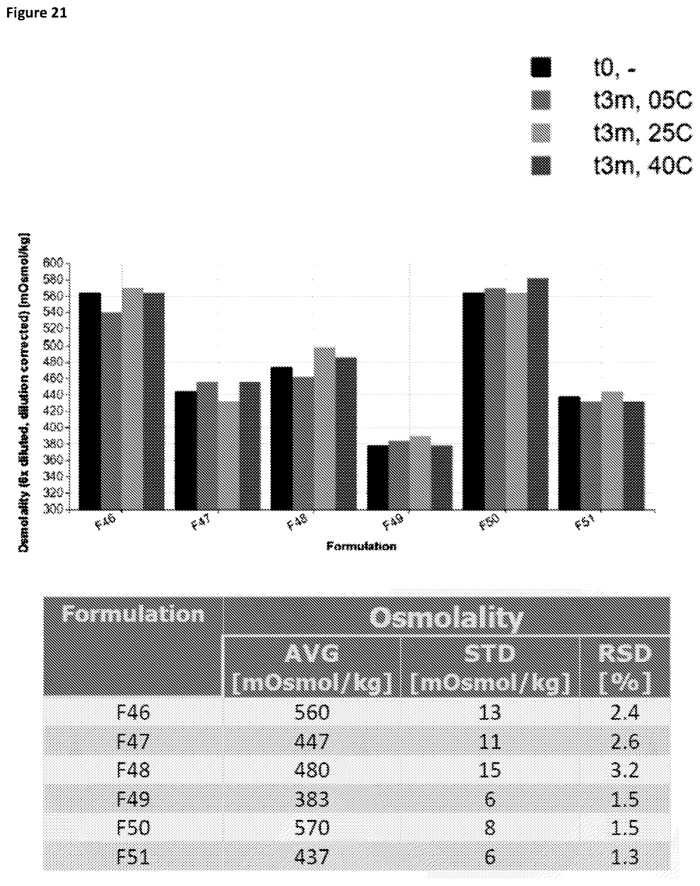

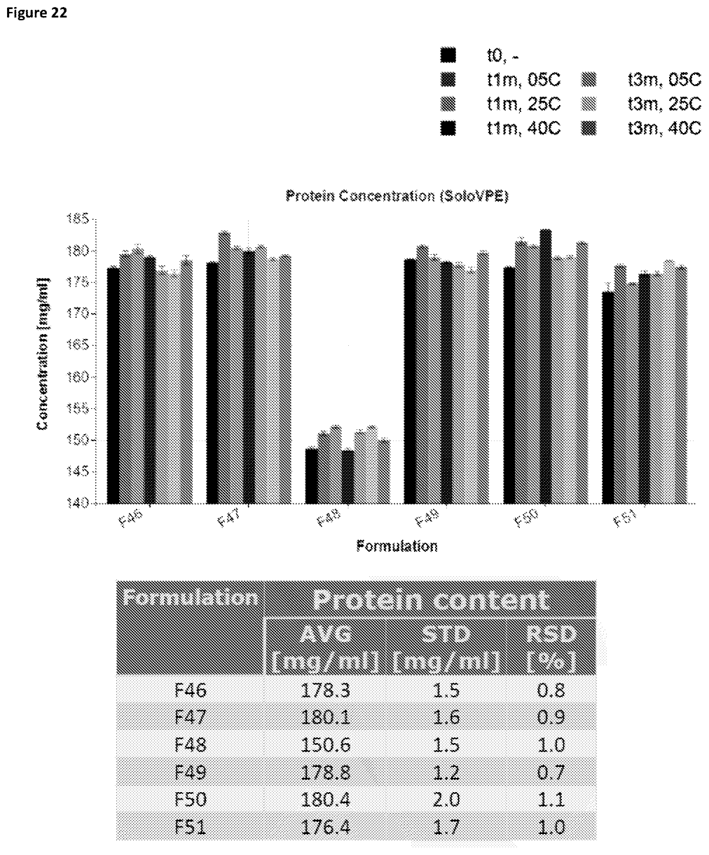

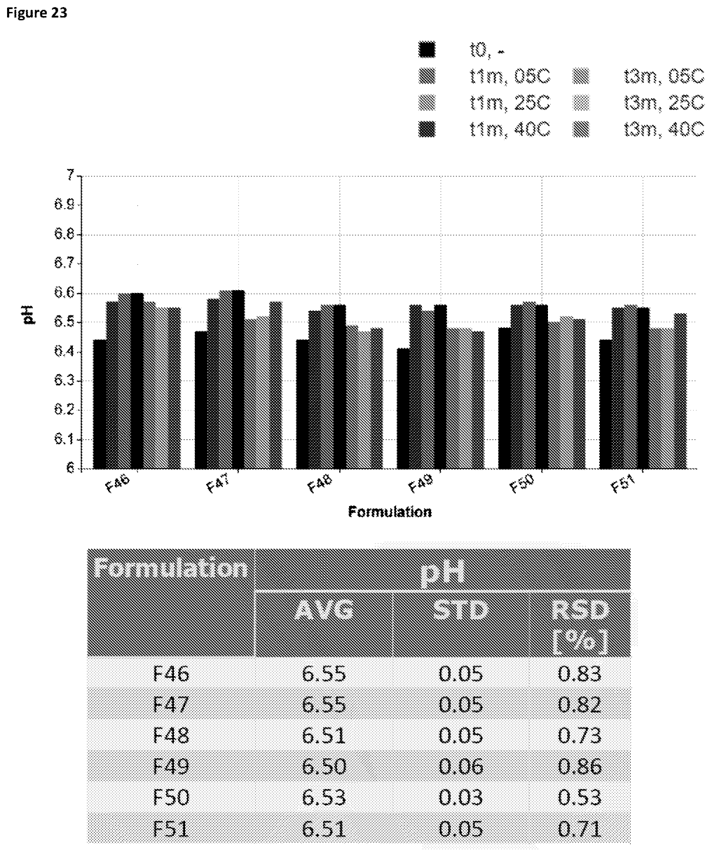

FIG. 23 shows the pH results for the 6 formulations. The differences observed were within the expected method variability.

FIG. 24 shows results of the visual inspection of 6 formulations. In general, the formulations appeared opalescent and with the exception of F49 at 40° C., the particles were at the limit of being visible. This suggests that particle formation was minimal.

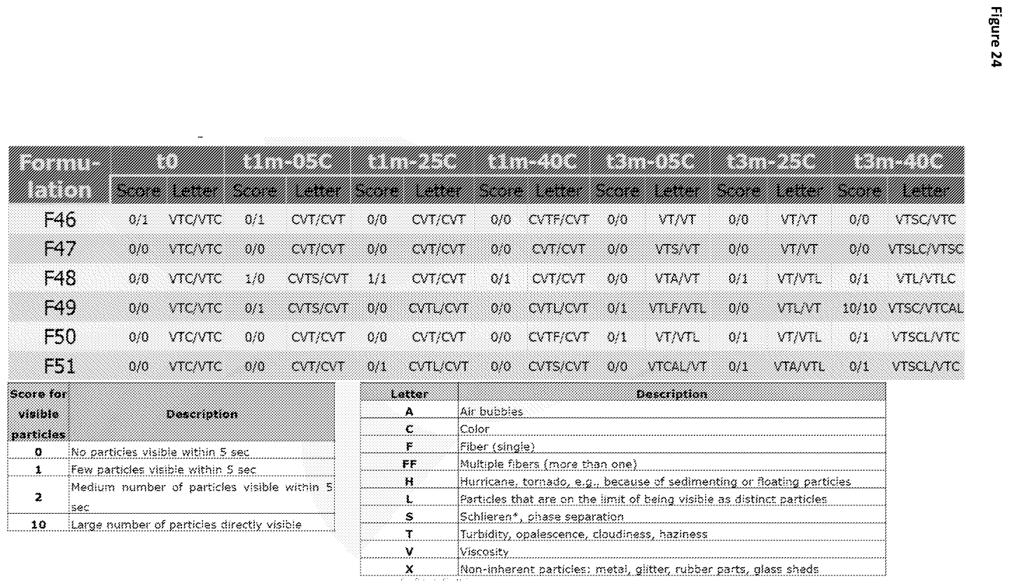

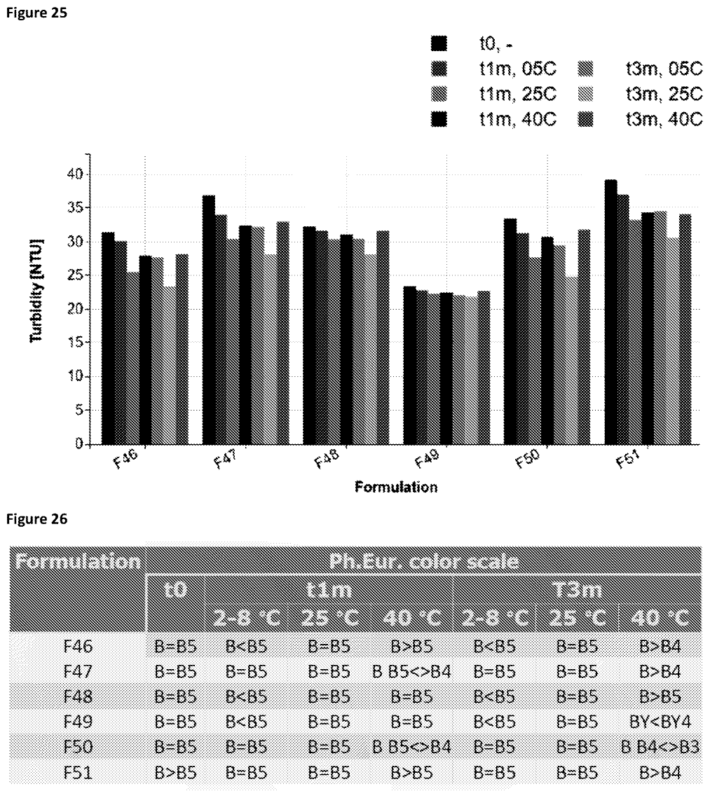

FIG. 25 shows turbidity results for the 6 formulations. The results suggest that the turbidity of the formulations was comparable between the Day 0, 1-month and 3-month samples. The lowest turbidity values were observed for the samples stored at 25° C.

FIG. 26 shows the colour change results for the 6 formulations. With the exception of F49 at 40° C., the rest of formulations remained within the brown spectrum (B5) after 3 months storage. No change in colouration was observed between the 1-month and 3-month samples stored at 2-8° C. or 25° C. Conversely, there was a small change in colouration after storage for 3 months at 40° C. compared to 1 month of storage at 40° C.

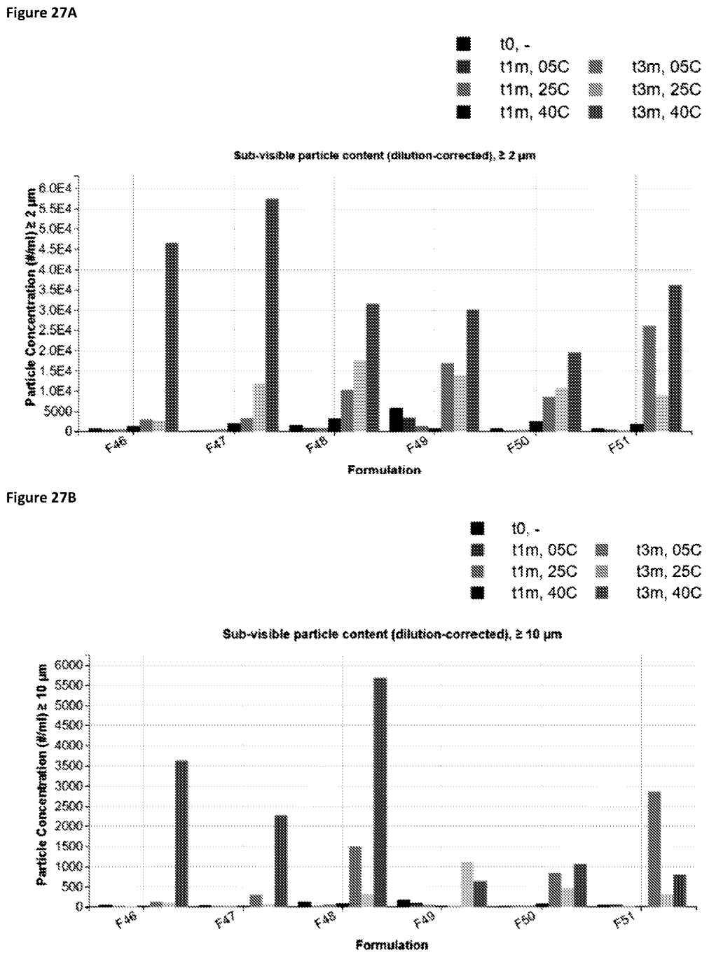

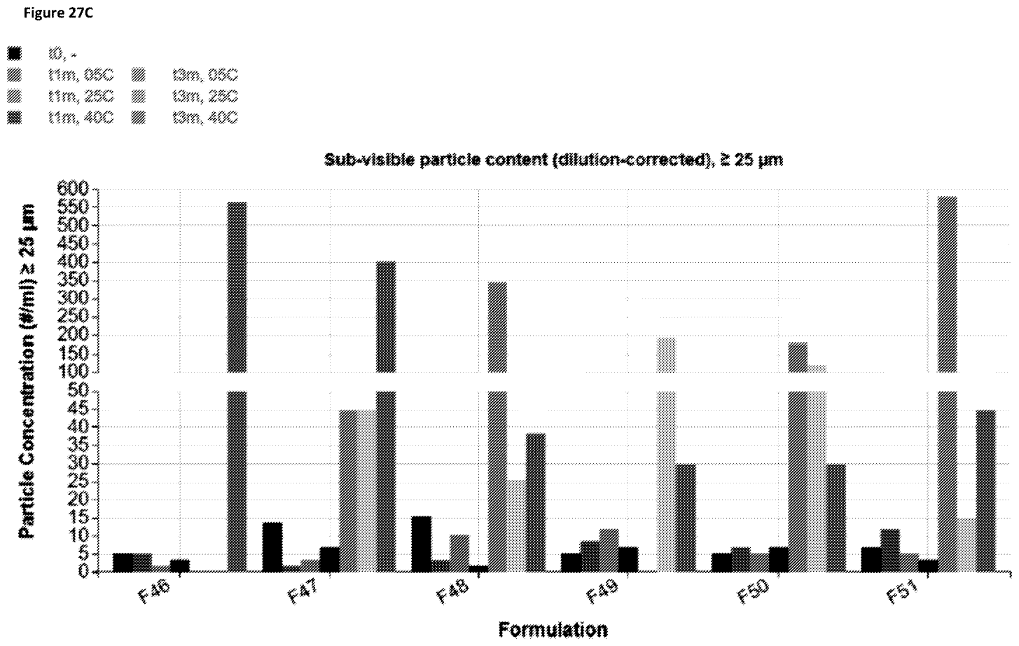

FIGS. 27A to 27C show the results of the subvisible particle content experiments performed using micro-flow imaging (MFI). An increased particle content of size≥2 μm after 3 months was observed in all samples, in particular the samples stored at 40° C.

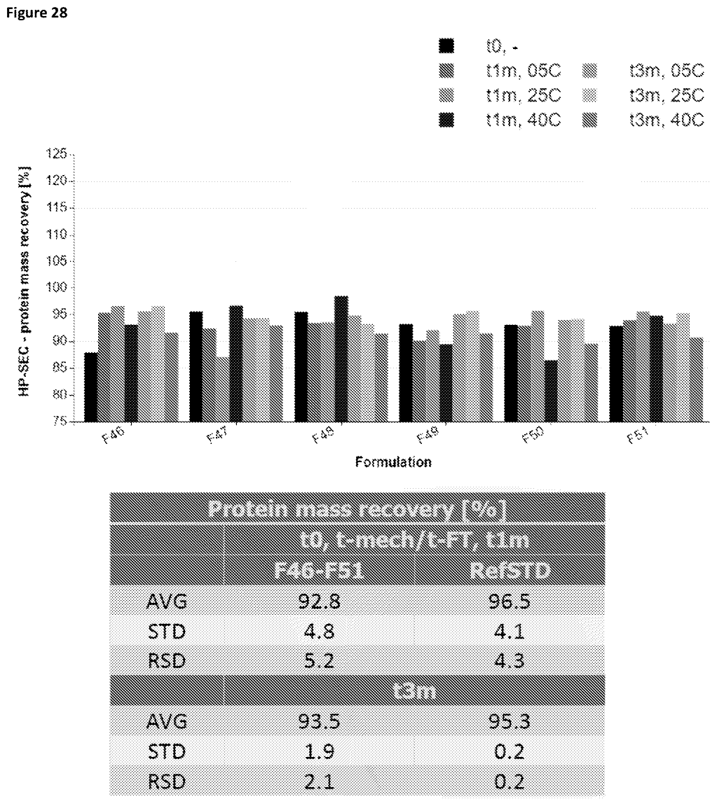

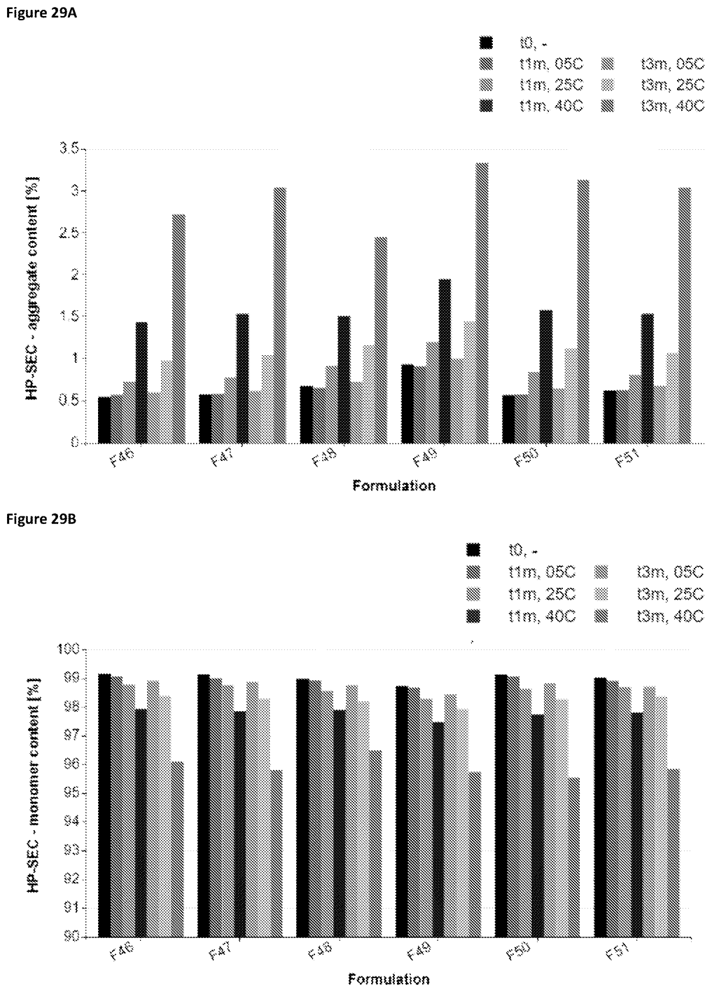

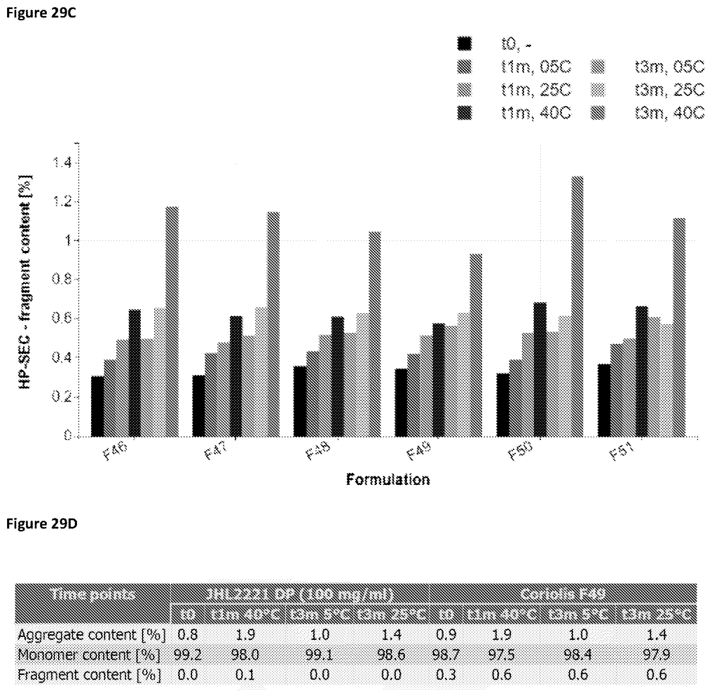

FIGS. 28 and 29A to 29D show the results of experiments conducted using HP-SEC. Details of the instrument and parameters used are as follows:

Instrument: Dionex Ultimate 3000; Column: Waters Xbridge protein BEH SEC, 7.8×300 mm

Detection: UV at 280 nm; Sample preparation: Dilution to 1 mg/ml (intermediate dilution 50 mg/ml) in 20 mM histidine-HCl at pH 6.5; Measurement: n=1

FIG. 28 suggest that the % protein mass recovery of all the formulations stored at 40° C. for 3 months is slightly lower compared to the formulations stored at 5° C. or 25° C. Nonetheless, there did not appear to be a significant difference between the formulations. The results in FIGS. 29A to 29C indicate that there was a similar loss of monomer content, increase of high molecular weight and fragment content across the 6 formulations. Nonetheless, the loss or monomer was relatively low-almost no loss after 3 months at 5° C., ≤1% loss after 3 months at 25° C., ≤4% loss after 3 months at 40° C. A higher aggregate content was observed for F49 compared to other formulations. FIG. 29D demonstrates that the intact protein % concentration measured using SEC for Formulation F49 was very similar to that of the 100 mg/ml eblasakimab formulation.

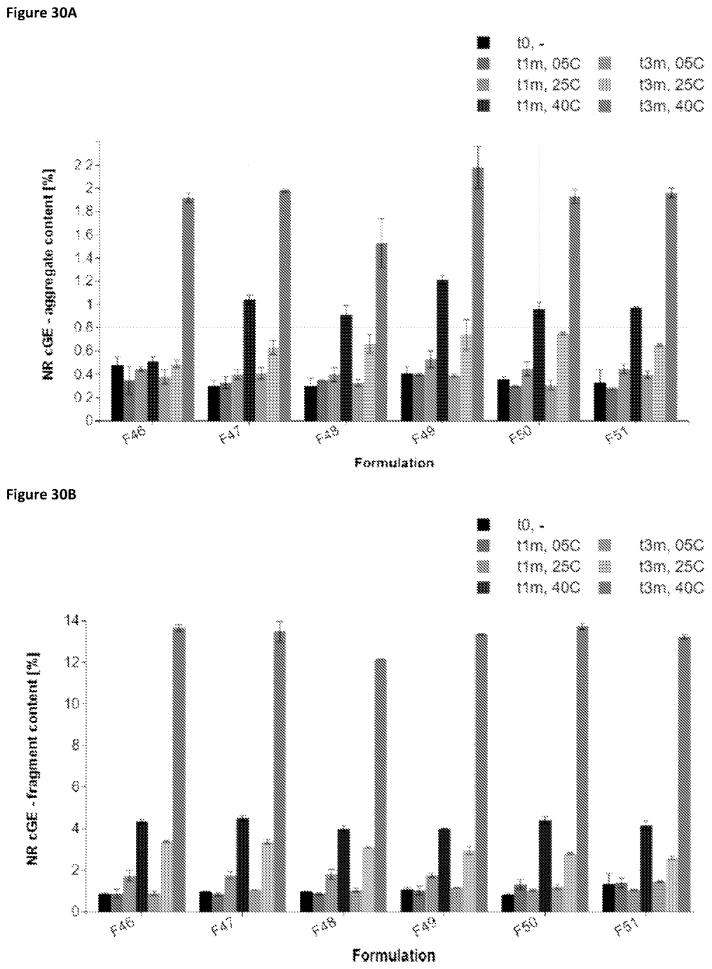

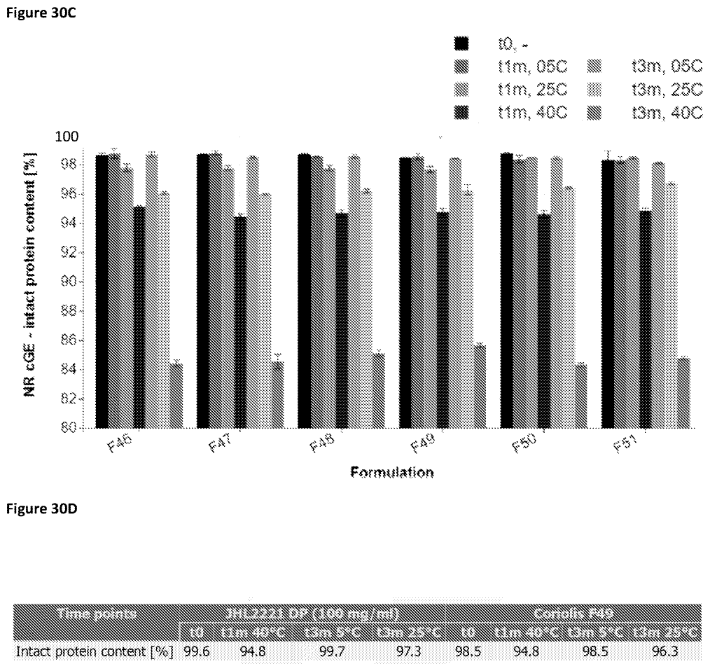

FIGS. 30A to 30D show the results of the experiments conducted using capillary gel electrophoresis (cGE) for the non-reduced samples. Details of the instrument and parameters used are as follows:

Instrument: SCIEX PA 800 plus equipped with a 30.2 cm bare-fused silica capillary. Sample preparation: sample dilution with 20 mM histidine in a two-step process (target concentration 5 mg/ml). Measurement: samples split into 2 runs on separate days (WP 2 day-to-day repeatability=0.8%), n=2

FIGS. 30A to 30C suggest that there was almost no loss of intact protein after 3 months at 5° C., a loss of less than 3% after 3 months at 25° C., and a loss of less then 15% after 3 months at 40° C., primarily due to fragmentation. FIG. 30D demonstrates that the intact protein % concentration measured using cGE for Formulation F49 was very similar to that of the 100 mg/ml eblasakimab formulation.

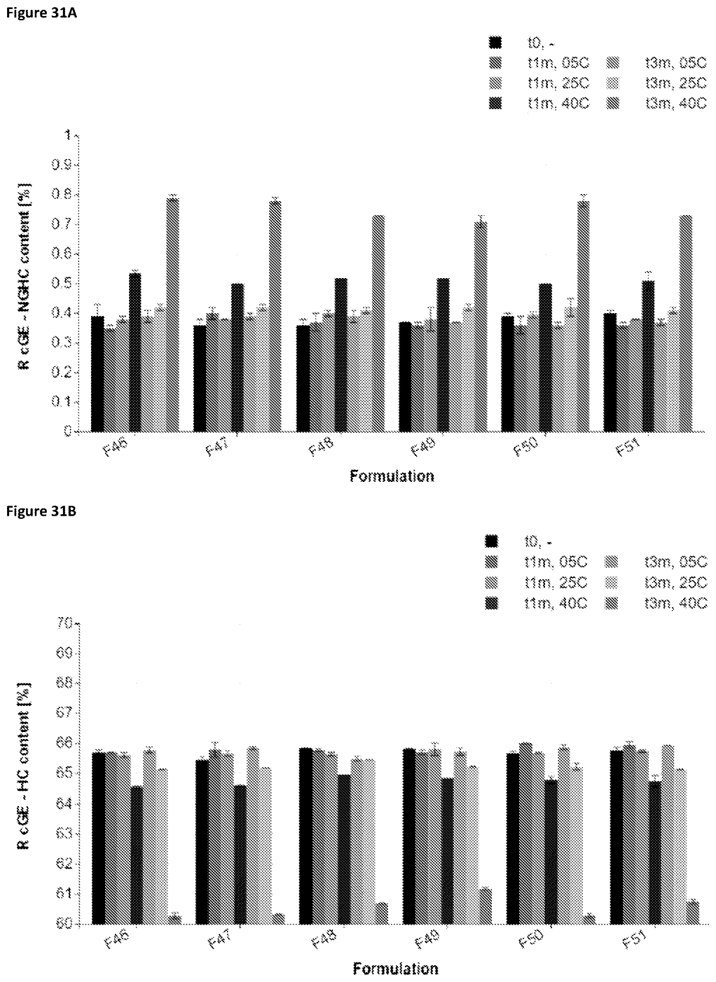

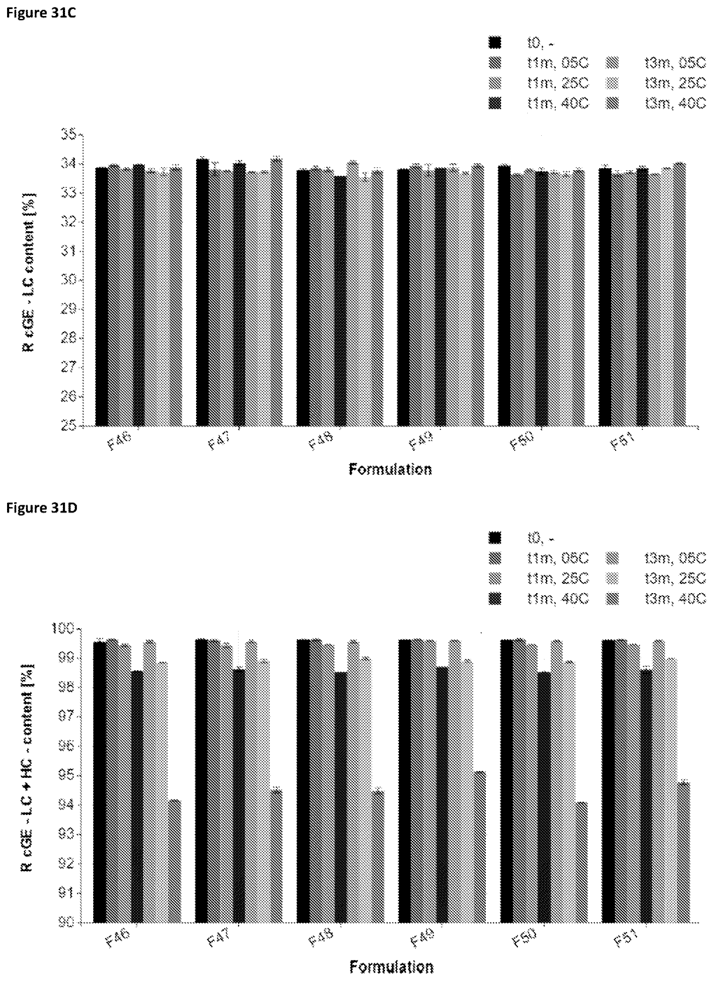

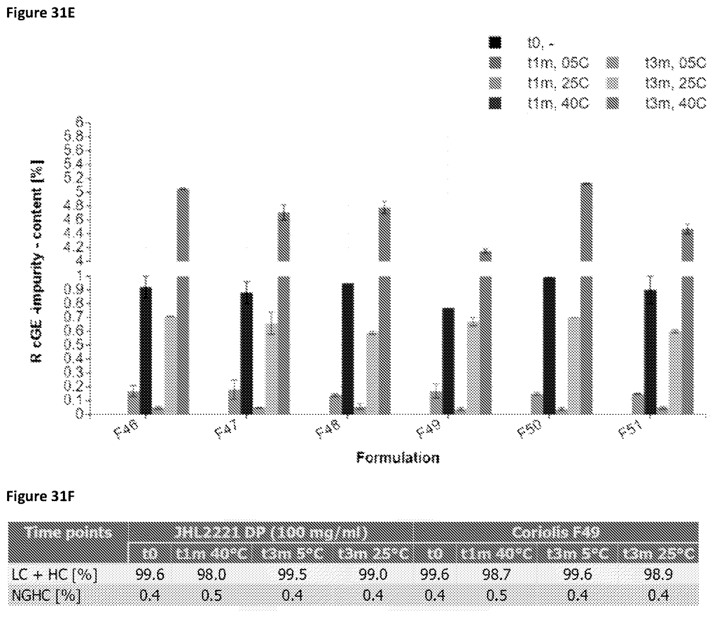

FIGS. 31A to 31F show the results of the experiments conducted using capillary gel electrophoresis (cGE) for the reduced samples.

FIGS. 31A to 31D indicate that there was no significant change in relative light chain (LC) content. There was a slight decrease in heavy chain (HC) content after 3 months at 25° C. and 40° C. When LC and HC content was considered together (FIG. 31D), the results suggest that LC+HC content was ≥99% after 3 months at 5° C. and >98% at 25° C. FIG. 31E shows the impurity levels after 1 month and 3 months storage. From the results, the inventors believe it is likely that the decrease in relative HC content after 3 months is partly due to deglycosylation of the reduced samples, and also the increasing impurity levels with time. This trend was seen across the various formulations.

FIG. 31F demonstrates that the LC+HC % and NGHC % measured for Formulation F49 was very similar to that of the 100 mg/ml eblasakimab formulation.

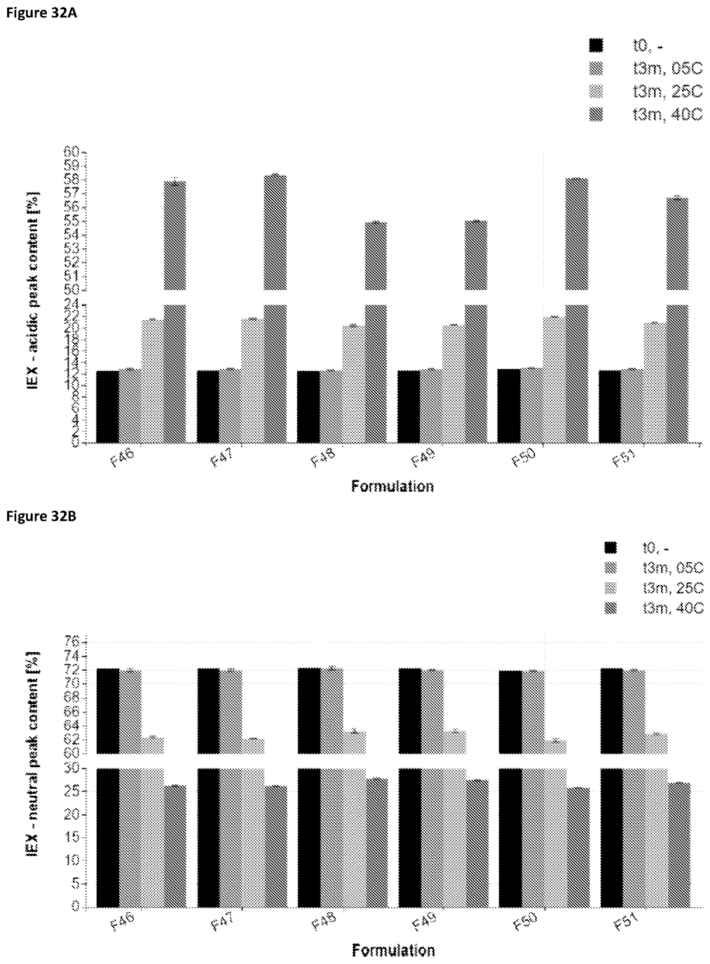

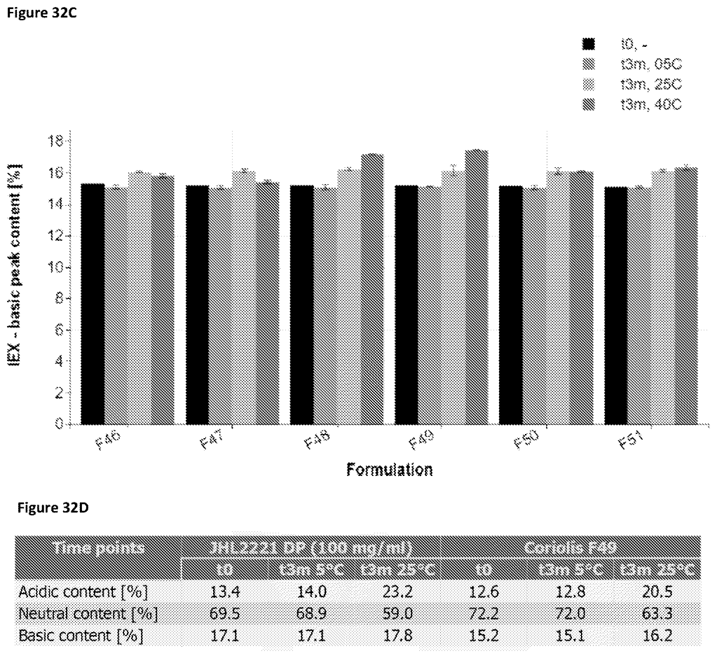

FIGS. 32A to 32D show the results of the ion-exchange chromatography (IEX) experiments. Details of the instrument and parameters used are as follows:

Instrument: Vanquish UHPLC; Column: ProPac WCX-10, 4.0×250 mm, 10 μm; Detection: UV at 280 nm; Sample preparation: sample dilution to 1 mg/ml (intermediate dilution 50 mg/ml) in 20 mM MES, pH 7.0; Measurement: n=2

FIGS. 32A to 32C indicate that there was a similar loss of neutral species, and a similar increase of acidic and basic species for all the formulations over the 3 months storage. There was almost no loss of neutral species after 3 months at 5° C., less than 10% loss of neutral species after 3 months at 25° C., and less than 45% loss of neutral species after 3 months at 40° C. FIG. 32D demonstrates that the acidic content, neutral content and basic content measured for Formulation F49 was very similar to that of the 100 mg/ml eblasakimab formulation.

In summary, the 3-month interim stability data was very promising—the data suggest that all 6 formulations tested had good stability at 3 months. Based on the results, formulation F46 was selected as the primary formulation going forward.

1-20. (canceled)

21. A high concentration antibody formulation, said formulation comprising:
150 to 210 mg/ml of an anti-IL-13R antibody or antigen binding fragment thereof;
170 to 260 mM of arginine;
20 to 50 mM histidine buffer;
0.01-0.03% of a non-ionic surfactant; and
wherein the pH of the formulation is in the range 6.0 to 7.0; and
wherein the anti-IL-13R antibody or antigen binding fragment thereof comprises:
a VH CDR1 comprising SEQ ID NO: 1,
a VH CDR2 comprising SEQ ID NO: 2,
a VH CDR3 comprising SEQ ID NO: 3,
a VL CDR1 comprising SEQ ID NO: 4,
a VL CDR2 comprising SEQ ID NO: 5, and
a VL CDR3 comprising SEQ ID NO: 6.

22. The formulation according to claim 21, wherein the anti-IL-13R antibody comprises a VH domain comprising an amino acid sequence shown in SEQ ID NO: 7 or a sequence at least 95% identical thereto and a VL domain comprising an amino acid sequence shown in SEQ ID NO: 8 or a sequence at least 95% identical thereto.

23. The formulation according to claim 21, comprising 150, 175 or 200 mg/ml of an anti-IL-13R antibody or antigen binding fragment thereof.

24. The formulation according to claim 21, comprising 175 mM, 200 or 250 mM arginine.

25. The formulation according to claim 21, comprising 260 mM arginine.

26. The formulation according to claim 21, wherein the arginine is Arg-HCl or Arg-Glu.

27. The formulation according to claim 21, comprising 20 mM or 50 mM histidine buffer.

28. The formulation according to claim 21, comprising 20 mM histidine buffer.

29. The formulation according to claim 21, comprising 0.02% w/w of a non-ionic surfactant.

30. The formulation according to claim 21, comprising 0.02% w/w of polysorbate 20.

31. The formulation according to claim 21, wherein the pH is 6.0, 6.5 or 7.0.

32. The formulation according to claim 21, wherein the pH is 6.5.

33. The formulation according to claim 21, wherein the formulation comprises a further amino acid.

34. The formulation according to claim 21, wherein the formulation comprises phenylalanine.

35. The formulation according to claim 21, wherein the formulation comprises 50, 75 or 80 mM phenylalanine.

36. The formulation according to claim 21, wherein the formulation further comprises CaCl₂).

37. The formulation according to claim 21, wherein the formulation further comprises 50 to 200 mM of a sugar.

38. The formulation according to claim 21, wherein the formulation comprises 180 mM of sucrose.

39. A high concentration antibody formulation according to claim 21, comprising:
175 mg/ml of an anti-IL-13R antibody or antigen binding fragment thereof;
250 mM of arginine;
20 mM histidine buffer;
0.02% of a non-ionic surfactant;
75 mM phenylalanine; and
wherein the pH of the formulation is 6.5.

40. A method of treating atopic dermatitis, comprising administering a therapeutically effective amount of a formulation according to claim 21.
